SEQUENCE LISTING (7 sequences)

SEQ ID NO: 1 - RNA, 4180 nt, Homo sapiens
  misc_feature 1..4180: note = Description of sequence: mRNA transcript encoding furin
  source 1..4180: mol_type = mRNA
gcggggaagcagcagcggccaggatgaatcccaggtgctctggagctggatggtgaaggtcggcactcttcaccctcccgagccctgcccgtctcggccccatgcccccaccagtcagccccgggccacaggcagtgagcaggcacctgggagccgaggccctgtgaccaggccaaggagacgggcgctccagggtcccagccacctgtcccccccatggagctgaggccctggttgctatgggtggtagcagcaacaggaaccttggtcctgctagcagctgatgctcagggccagaaggtcttcaccaacacgtgggctgtgcgcatccctggaggcccagcggtggccaacagtgtggcacggaagcatgggttcctcaacctgggccagatcttcggggactattaccacttctggcatcgaggagtgacgaagcggtccctgtcgcctcaccgcccgcggcacagccggctgcagagggagcctcaagtacagtggctggaacagcaggtggcaaagcgacggactaaacgggacgtgtaccaggagcccacagaccccaagtttcctcagcagtggtacctgtctggtgtcactcagcgggacctgaatgtgaaggcggcctgggcgcagggctacacagggcacggcattgtggtctccattctggacgatggcatcgagaagaaccacccggacttggcaggcaattatgatcctggggccagttttgatgtcaatgaccaggaccctgacccccagcctcggtacacacagatgaatgacaacaggcacggcacacggtgtgcgggggaagtggctgcggtggccaacaacggtgtctgtggtgtaggtgtggcctacaacgcccgcattggaggggtgcgcatgctggatggcgaggtgacagatgcagtggaggcacgctcgctgggcctgaaccccaaccacatccacatctacagtgccagctggggccccgaggatgacggcaagacagtggatgggccagcccgcctcgccgaggaggccttcttccgtggggttagccagggccgaggggggctgggctccatctttgtctgggcctcggggaacgggggccgggaacatgacagctgcaactgcgacggctacaccaacagtatctacacgctgtccatcagcagcgccacgcagtttggcaacgtgccgtggtacagcgaggcctgctcgtccacactggccacgacctacagcagtggcaaccagaatgagaagcagatcgtgacgactgacttgcggcagaagtgcacggagtctcacacgggcacctcagcctctgcccccttagcagccggcatcattgctctcaccctggaggccaataagaacctcacatggcgggacatgcaacacctggtggtacagacctcgaagccagcccacctcaatgccaacgactgggccaccaatggtgtgggccggaaagtgagccactcatatggctacgggcttttggacgcaggcgccatggtggccctggcccagaattggaccacagtggccccccagcggaagtgcatcatcgacatcctcaccgagcccaaagacatcgggaaacggctcgaggtgcggaagaccgtgaccgcgtgcctgggcgagcccaaccacatcactcggctggagcacgctcaggcgcggctcaccctgtcctataatcgccgtggcgacctggccatccacctggtcagccccatgggcacccgctccaccctgctggcagccaggccacatgactactccgcagatgggtttaatgactgggccttcatgacaactcattcctgggatgaggatccctctggcgagtgggtcctagagattgaaaacaccagcgaagccaacaactatgggacgctgaccaagttcaccctcgtactctatggcaccgcccctgaggggctgcccgtacctccagaaagcagtggctgcaagaccctcacgtccagtcaggcctgtgtggtgtgcgaggaaggcttctccctgcaccagaagagctgtgtccagcactgccctccagggttcgccccccaagtcctcgatacgcactatagcaccgagaatgacgtggagaccatccgggccagcgtctgcgccccctgccacgcctcatgtgccacatgccaggggccggccctgacagactgcctcagctgccccagccacgcctccttggaccctgtggagcagacttgctcccggcaaagccagagcagccgagagtccccgccacagcagcagccacctcggctgcccccggaggtggaggcggggcaacggctgcgggcagggctgctgccctcacacctgcctgaggtggtggccggcctcagctgcgccttcatcgtgctggtcttcgtcactgtcttcctggtcctgcagctgcgctctggctttagttttcggggggtgaaggtgtacaccatggaccgtggcctcatctcctacaaggggctgccccctgaagcctggcaggaggagtgcccgtctgactcagaagaggacgagggccggggcgagaggaccgcctttatcaaagaccagagcgccctctgatgagcccactgcccaccccctcaagccaatcccctccttgggcactttttaattcaccaaagtatttttttatcttgggactgggtttggaccccagctgggaggcaagaggggtggagactgcttcccatcctaccctcgggcccacctggccacctgaggtgggcccaggaccagctggggcgtggggagggccgtaccccaccctcagcaccccttccatgtggagaaaggagtgaaacctttagggcagcttgccccggccccggccccagccagagttcctgcggagtgaagaggggcagcccttgcttgttgggattcctgacccaggccgcagctcttgcccttccctgtccctctaaagcaataatggtcccatccaggcagtcgggggctggcctaggagatatctgagggaggaggccacctctccaagggcttctgcaccctccaccctgtcccccagctctggtgagtcttggcggcagcagccatcataggaagggaccaaggcaaggcaggtgcctccaggtgtgcacgtggcatgtggcctgtggcctgtgtcccatgacccacccctgtgctccgtgcctccaccaccactggccaccaggctggcgcagccaaggccgaagctctggctgaaccctgtgctggtgtcctgaccaccctcccctctcttgcacccgcctctcccgtcagggcccaagtccctgttttctgagcccgggctgcctgggctgttggcactcacagacctggagcccctgggtgggtggtggggaggggcgctggcccagccggcctctctggcctcccacccgatgctgctttcccctgtggggatctcaggggctgtttgaggatatattttcactttgtgattatttcactttagatgctgatgatttgtttttgtatttttaatgggggtagcagctggactacccacgttctcacacccaccgtccgccctgctcctccctggctgccctggccctgaggtgtgggggctgcagcatgttgctgaggagtgaggaatagttgagccccaagtcctgaagaggcgggccagccaggcgggctcaaggaaagggggtcccagtgggaggggcaggctgacatctgtgtttcaagtggggctcgccatgccgggggttcataggtcactggctctccaagtgccagaggtgggcaggtggtggcactgagcccccccaacactgtgccctggtggagaaagcactgacctgtcatgcccccctcaaacctcctcttctgacgtgccttttgcacccctcccattaggacaatcagtcccctcccatctgggagtccccttttcttttctaccctagccattcctggtacccagccatctgcccaggggtgccccctcctctcccatccccctgccctcgtggccagcccggctggttttgtaagatgctgggttggtgcacagtgatttttttcttgtaatttaaacaggcccagcattgctggttctatttaatggacatgagataatgttagaggttttaaagtgattaaacgtgcagactatgcaaaccag

SEQ ID NO: 2 - RNA, 245 nt, synthetic construct
  misc_feature 1..245: note = Description of sequence: bi-shRNAfurin
  source 1..245: mol_type = other RNA
gatcctgctgttgacagtgagcgcggagaaaggagtgaaaccttagtgaagccacagatgtaaggtttcactcctttctccttgcctactgcctcggaagcagctcactacattactcagctgttgacagtgagcgcggagaaagatatgaaaccttagtgaagccacagatgtaaggtttcactcctttctccttgcctactgcctcggaagcttaataaaggatcttttattttcattggatc

SEQ ID NO: 3 - RNA, 21 nt, synthetic construct
  misc_feature 1..21: note = Description of sequence: Passenger strand in first stem-loop structure in bi-shRNAfurin
  source 1..21: mol_type = other RNA
ggagaaaggagtgaaacctta

SEQ ID NO: 4 - RNA, 21 nt, synthetic construct
  misc_feature 1..21: note = Description of sequence: Guide strand in both first and second stem-loop structures in bi-shRNAfurin
  source 1..21: mol_type = other RNA
taaggtttcactcctttctcc

SEQ ID NO: 5 - RNA, 21 nt, synthetic construct
  misc_feature 1..21: note = Description of sequence: Passenger strand in second stem-loop structure in bi-shRNAfurin
  source 1..21: mol_type = other RNA
ggagaaagatatgaaacctta

SEQ ID NO: 6 - RNA, 15 nt, synthetic construct
  misc_feature 1..15: note = Description of sequence: miR-30a loop
  source 1..15: mol_type = other RNA
gtgaagccacagatg

SEQ ID NO: 7 - DNA, 5140 bp, synthetic construct
  misc_feature 1..5140: note = Description of sequence: Vector plasmid
  source 1..5140: mol_type = other DNA
ggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattccgctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctaaaaaaaaaaaaaaaaaaaaaaacggaattcacgtgggcccggtaccgtatactctagaagatctggcagcggagagggcagaggaagtcttctaacatgcggtgacgtggaggagaatcccggccctaggatgtctagagcggccgcggatcctgctgttgacagtgagcgcggagaaaggagtgaaaccttagtgaagccacagatgtaaggtttcactcctttctccttgcctactgcctcggaagcagctcactacattactcagctgttgacagtgagcgcggagaaagatatgaaaccttagtgaagccacagatgtaaggtttcactcctttctccttgcctactgcctcggaagcttaataaaggatcttttattttcattggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctatt

(19) United States
(12) Patent Application Publication
(10) Pub. No.: US 2025/0003010 A1
(43) Pub. Date: Jan. 2, 2025

(54) METHODS FOR TREATMENT RESPONSE TO CANCERS

(71) Applicant: Gradalis, Inc., Carrollton, TX (US)

(72) Inventors: Laura Nejedlik, Dallas, TX (US); Min Tang, Dallas, TX (US); John Joseph Nemunaitis, Dallas, TX (US)

(21) Appl. No.: 18/709,783

(22) PCT Filed: Nov. 21, 2022
(86) PCT No.: PCT/US2022/050542

§ 371 Date: May 13, 2024

Related U.S. Application Data
(60) Provisional application No. 63/388,140, filed on Jul. 11, 2022.
(60) Provisional application No. 63/284,545, filed on Nov. 30, 2021.

(51) Int. Cl. C12Q 1/6886; A61K 38/19; A61K 48/00; A61P 35/00; C12N 15/113

(52) U.S. Cl. CPC C12Q 1/6886; A61K 38/193; A61K 48/005; A61P 35/00; C12N 15/113; C12Q 2600/106; C12Q 2600/158

(57) ABSTRACT

Disclosed herein are methods for predicting the responsiveness of a cancer in a subject to a therapy and methods for treating the cancer by determining the expression level of one or more genes selected from the group consisting of ENTPD1, CCL13, CD79B, and MRC1 in the subject.

### CROSS-REFERENCES TO RELATED APPLICATIONS

This application claims priority to U.S. Provisional Application No. 63/284,545, filed Nov. 30, 2021, and U.S. Provisional Application No. 63/388,140, filed Jul. 11, 2022, the disclosures of which are hereby incorporated by reference in their entirety for all purposes.

### BACKGROUND OF THE DISCLOSURE

Vigil® is a novel autologous tumor cell immunotherapy, which is constructed from harvested malignant tissue. It incorporates a multigenic plasmid encoding the human immune-stimulatory GMCSF gene and a bifunctional short-hairpin RNA construct, which specifically knocks down the proprotein convertase furin and its downstream targets TGFβ1 and TGFβ2. It is also designed to facilitate both cancer-associated antigen and neoantigen expression, upregulate MHC-II and enhance bone-marrow derived dendritic cell maturation, thereby augmenting the afferent immune response, and generating a systemic antitumor effect.

The VITAL study (NCT02346747) was a Phase IIb double-blind, placebo-controlled trial involving women 18 years and older with Stage III/IV high-grade serous, endometroid or clear cell ovarian cancer (OC) in clinical complete response (CCR) following carboplatin and paclitaxel induction chemotherapy. Results in a subset of 67 patients with BRCA1/2-wildtype (wt) OC showed improved relapse free survival (RFS; HR=0.51, p=0.02) and overall survival (OS; HR=0.49, p=0.049) compared to placebo. Moreover, ad hoc analysis of a subset of 45 patients with homologous repair proficient (HRP) tumors by Myriad MyChoice CDx (Myriad Genetics, Salt Lake City, UT) also showed improvement in RFS and OS (HR=0.39, p=0.007 and HR=0.34, p=0.019, respectively). Long term follow-up confirmed a durable survival effect. Three-year survival proportion from time of procurement was 83% for Vigil® and 40% for placebo (p=0.0006). A correlation of systemic immune response to Vigil® clinical benefit was noted using ELISPOT assay.

Contemporary clinical management of oncology patients is increasingly being guided by predictive molecular and phenotypic profiling in order to optimize the use of targeted- and immuno-therapeutics, e.g., tumor mutational burden (TMB), MMR, PD-1, and PD-L1. However, the use of predictive biomarkers for immunotherapy in OC has not consistently translated into clinical benefit despite documented responses in some patients. Although genomically unstable, OC is not mutationally driven, thus the clinical efficacy of immunotherapy in this disease has been dismal (<10% which generally correlates with high TMB, a presumptive marker of neoantigen content), represented by several failed phase III clinical trials.

Nevertheless, we have studied patient subpopulations most sensitive to Vigil® therapy based on molecular profile using NanoString assessment, and demonstrated that TIS^{high} score (tumor inflammation score) and MHC-II expression correlated with ELISPOT reactivity and clinically to OS and RFS. Likewise, using NanoString technology to assess OS and RFS in patients enrolled in the VITAL study, we showed marked benefit in patients with BRCA1/2-wt and HRP profiles and improved outcomes in patients whose tumors had mutant TP53 (p=0.0013). The current study explores the relationship of mRNA expression via NanoString analysis in harvested baseline tumor to RFS and OS in Vigil® treated patients from the VITAL study.

Current ovarian cancer treatments have shown limited efficacy in advanced stage patients. Treatments that increase a patient's immune system have shown efficacy, however accurately determining which patients will have benefit has been difficult. Therefore, there is a need to develop biomarkers that will predict which patients will respond to therapy.

This disclosure provides methods that evaluate tumor gene expression data to determine which patients will respond to Vigil® treatment and offers other advantages as well.

### BRIEF SUMMARY OF THE DISCLOSURE

Vigil® is a triple function immune therapy that modifies the patient's own tumor cells to activate the immune system. The present disclosure demonstrates that high expression of a gene known as ENTPD1/CD39 predicts a positive response to Vigil® therapy. In certain aspects, this method aids to prospectively refine which patients respond to Vigil. Additionally, this analysis is used in a broad application with other targeted therapies to identify patients responsive to therapy.

In one aspect, the gene expression of ENTPD1/CD39 demonstrates clinical significance as a presumptive predictor of Vigil® response versus placebo regardless of HRP status.

In one aspect, the disclosure provides a method for treating an individual having cancer, the method comprising:
profiling an expression level of one or more genes selected from the group consisting of ENTPD1, CCL13, CD79B, and MRC1, in a tumor obtained from said individual;
determining the presence of an elevated expression level of the one or more genes selected from the group consisting of ENTPD1, CCL13, CD79B, and MRC1 in the tumor obtained from said individual; and
administering to the individual a therapy comprising an expression vector having (i) a first insert comprising a nucleic acid sequence encoding a Granulocyte Macrophage Colony Stimulating Factor (GM-CSF) sequence; and (ii) a second insert, wherein the second insert encodes one or more bifunctional short hairpin RNAs (shRNA) capable of hybridizing to one of more regions of a mRNA transcript encoding furin, wherein at least one of the regions is selected from base sequences 300-318, 731-740, 1967-1991, 2425-2444, 2827-2851 or 2834-2852 of SEQ ID NO:1, thereby inhibiting furin expression via RNA interference, wherein each bifunctional short hairpin RNA comprises a first stem-loop structure that comprises an siRNA component and a second stem-loop structure that comprises a miRNA component and wherein the shRNA incorporates siRNA (cleavage dependent) and miRNA (cleavage-independent) motifs, and wherein the therapy improves the treatment response in the individual with the elevated expression level compared to a patient with a tumor having a low expression level of the gene.

In some embodiments of this aspect, the second insert comprises two stem-loop structures each with a miR-30a loop, and the first stem-loop structure has complete complementary guiding strand and passenger strand, while the second stem-loop structure has three basepair (bp) mismatches at positions 9 to 11 of the passenger strand. In particular embodiments, the second insert comprises a sequence having at least 90% (e.g., 91%, 92%, 93%, 94%, 95%, 96%, 97%, 98%, 99%, or 100%) identity to the sequence of SEQ ID NO:2. In certain embodiments, the second insert comprises the sequence of SEQ ID NO:2.

In another aspect, the disclosure provides a method for predicting responsiveness of an individual having or suspected of having cancer to a therapy comprising an expression vector having (i) a first insert comprising a nucleic acid sequence encoding a Granulocyte Macrophage Colony Stimulating Factor (GM-CSF) sequence; and (ii) a second insert, the method comprising:
profiling the expression level of one or more genes selected from the group consisting of ENTPD1, CCL13, CD79B, and MRC1, in a tumor obtained from said individual;
determining the presence of an elevated expression level of the one or more genes selected from the group consisting of ENTPD1, CCL13, CD79B, and MRC1; and
identifying the individual with the elevated expression level of the one or more genes as predicted to have an increased responsiveness to the therapy, compared to a patient with a tumor having a low expression of the gene,
wherein the second insert encodes one or more bifunctional short hairpin RNAs (shRNA) capable of hybridizing to one of more regions of a mRNA transcript encoding furin, wherein at least one of the regions is selected from base sequences 300-318, 731-740, 1967-1991, 2425-2444, 2827-2851 or 2834-2852 of SEQ ID NO:1, thereby inhibiting furin expression via RNA interference, wherein each bifunctional short hairpin RNA comprises a first stem-loop structure that comprises an siRNA component and a second stem-loop structure that comprises a miRNA component and wherein the shRNA incorporates siRNA (cleavage dependent) and miRNA (cleavage-independent) motifs.

In some embodiments of this aspect, the second insert comprises two stem-loop structures each with a miR-30a loop, and the first stem-loop structure has complete complementary guiding strand and passenger strand, while the second stem-loop structure has three basepair (bp) mismatches at positions 9 to 11 of the passenger strand. In particular embodiments, the second insert comprises a sequence having at least 90% (e.g., 91%, 92%, 93%, 94%, 95%, 96%, 97%, 98%, 99%, or 100%) identity to the sequence of SEQ ID NO:2. In certain embodiments, the second insert comprises the sequence of SEQ ID NO:2.

In still another aspect, the disclosure provides a method for selecting an individual having cancer to be subjected to a therapy comprising an expression vector having (i) a first insert comprising a nucleic acid sequence encoding a Granulocyte Macrophage Colony Stimulating Factor (GM-CSF) sequence; and (ii) a second insert, the method comprising:
profiling the expression level of one or more genes selected from the group consisting of ENTPD1, CCL13, CD79B, and MRC1, in a tumor obtained from said individual;
determining the presence of an elevated expression level of the one or more genes selected from the group consisting of ENTPD1, CCL13, CD79B, and MRC1; and
selecting the individual who is determined to have the elevated expression level of the one or more genes compared to a patient with a tumor having a low expression of the gene as an individual to be subjected to the therapy,
wherein the second insert encodes one or more bifunctional short hairpin RNAs (shRNA) capable of hybridizing to one of more regions of a mRNA transcript encoding furin, wherein at least one of the regions is selected from base sequences 300-318, 731-740, 1967-1991, 2425-2444, 2827-2851 or 2834-2852 of SEQ ID NO:1, thereby inhibiting furin expression via RNA interference, wherein each bifunctional short hairpin RNA comprises a first stem-loop structure that comprises an siRNA component and a second stem-loop structure that comprises a miRNA component and wherein the shRNA incorporates siRNA (cleavage dependent) and miRNA (cleavage-independent) motifs, and wherein the therapy improves the treatment response in the individual with the elevated expression level compared to a patient with a tumor having a low expression level of the gene.

In some embodiments of this aspect, the second insert comprises two stem-loop structures each with a miR-30a loop, and the first stem-loop structure has complete complementary guiding strand and passenger strand, while the second stem-loop structure has three basepair (bp) mismatches at positions 9 to 11 of the passenger strand. In particular embodiments, the second insert comprises a sequence having at least 90% (e.g., 91%, 92%, 93%, 94%, 95%, 96%, 97%, 98%, 99%, or 100%) identity to the sequence of SEQ ID NO:2. In certain embodiments, the second insert comprises the sequence of SEQ ID NO:2.

In some embodiments, the GM-CSF is a human GM-CSF sequence.

In some embodiments, the expression vector further comprises a promoter. In some embodiments, the promoter is a cytomegalovirus (CMV) mammalian promoter. In some embodiments, the expression vector further comprises a CMV enhancer sequence and a CMV intron sequence.

In some embodiments, the expression vector further comprises a nucleic acid sequence encoding a picornaviral 2A ribosomal skip peptide between the first and the second nucleic acid inserts. In some embodiments, the expression vector is within an autologous cancer cell that is transfected with the expression vector. In some embodiments, the autologous cancer cell is administered to the individual as a dose of about 1×10⁶ cells to about 5×10⁷ cells. In some embodiments, the autologous cancer cell is administered to the individual once a month. In some embodiments, the autologous cancer cell is administered to the individual from 1 to 12 months. In some embodiments, the autologous cancer cell is administered to the subject by intradermal injection.

In some embodiments, the first insert and the second insert are operably linked to the promoter.

In some embodiments of the aspects described herein, the cancer is an HRD-negative, wild-type BRCA1/2 cancer.

In some embodiments of the aspects described herein, the cancer is selected from the group consisting of a solid tumor cancer, ovarian cancer, adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, prostate cancer, sarcoma, stomach cancer, uterine cancer, thyroid cancer, and a hematological cancer. In particular embodiments, the solid tumor cancer is selected from the group consisting of endometrial cancer, biliary cancer, bladder cancer, liver hepatocellular carcinoma, gastric/esophageal cancer, ovarian cancer, melanoma, breast cancer, pancreatic cancer, colorectal cancer, glioma, non-small-cell lung carcinoma, prostate cancer, cervical cancer, kidney cancer, thyroid cancer, a neuroendocrine cancer, small cell lung cancer, a sarcoma, head and neck cancer, brain cancer, clear cell renal cell carcinoma, skin cancer, endocrine tumor, thyroid cancer, tumor of unknown origin, and a gastrointestinal stromal tumor.

In particular embodiments, the cancer is ovarian cancer. In particular embodiments, the cancer is breast cancer. In particular embodiments, the cancer is melanoma. In particular embodiments, the cancer is lung cancer. In particular embodiments, the cancer is ovarian cancer and the method prevents or delays relapse of a substantially eradicated ovarian cancer. In certain embodiments, the substantially eradicated ovarian cancer is Stage III or Stage IV ovarian cancer.

In some embodiments of the aspects described herein, the subject received an initial therapy. In particular embodiments, the initial therapy comprises debulking surgery, chemotherapy, or the combination thereof. In some embodiments, the chemotherapy comprises administering a platinum-based drug and a taxane. In particular embodiments, the platinum-based drug comprises carboplatin. In particular embodiments, the taxane comprises paclitaxel.

In some embodiments of the aspects described herein, the methods further comprise administering an additional therapeutic agent. In some embodiments, the additional therapeutic agent is a member selected from the group consisting of an angiogenesis inhibitor, a PARP inhibitor, and a checkpoint inhibitor to the individual.

In still yet another embodiment, the disclosure provides a method for identifying genes that are predictive of responsiveness of an individual having or suspected of having cancer to a therapy, the method comprising:
(a) applying a univariate Cox model to Z-scores of a plurality of genes as a continuous variable for both overall survival (OS) and relapse free survival (RFS) in a cohort of patients treated with the therapy to generate a p-value and a corresponding 95% confidence interval (CI) for each of the plurality of genes;
(b) determining whether each gene identified in step (a) is predictive of a treatment advantage of the therapy by analyzing data of both the therapy cohort and a cohort of patients treated with a placebo by using a Cox proportional hazards model to determine if the interaction term between the gene and the treatment cohort was significant to yield predictive genes; and
(c) applying a further univariant Cox model to the identified predictive genes in step (b) in the treated cohort to further refine identification of relevant genes.

In some embodiments of the aspects described herein, step (c) comprises applying forward selection and backward elimination methodology to identify predictive genes associated with OS and RFS in the treated cohort.

In some embodiments, the method further comprises manually identify the predictive genes by dropping the covariates with p value greater than 0.01.

In some embodiments of the aspects described herein, the covariates are dropped one at a time.

In some embodiments of the aspects described herein, the method further comprises determining the expression levels of the identified genes in the individual.

In some embodiments of the aspects described herein, the therapy is Vigil®.

In some embodiments of the aspects described herein, the plurality of genes in step (a) is between 500 and 1000 genes.

In some embodiments of the aspects described herein, plurality of genes is about 750 genes.

In some embodiments of the aspects described herein, in the cohort of treated Vigil patients in step (b), 13 genes are determined to be statistically significant in OS and RFS, with 4 common genes.

These and other aspects, objects and embodiments will become more apparent when read with detailed descriptions and figures that follow.

### BRIEF DESCRIPTION OF THE DRAWINGS

FIG. 1 illustrates one embodiment of the flow of patients through the VITAL trial.

FIG. 2 illustrates one embodiment of a flow chart of all patients' analysis. Analyzed both with genes as raw continuous data and with genes dichotomized. Genes were selected if the interaction term was significant in both analyses. 5% alpha was used unless noted.

FIGS. 3A-3D illustrate overall survival (OS) Kaplan Meier (KM) curves of ENTPD1, CCL13, CD79B and MRC1 expression<median in Vigil® (n=24, 23, 21, 26 respectively) and placebo (n=21, 22, 24, 19 respectively) and ENTPD1, CCL13, CD79B and MRC1 expression≥median in Vigil® (n=23, 24, 26, 21, respectively) and placebo (n=23, 22, 20, 25, respectively) treated patients.

FIGS. 4A-4D illustrate Recurrence Free Survival Kaplan Meier of ENTPD1, CCL13, CD79B and MRC1 expression<median in Vigil® (n=24, 23, 21, 26 respectively) and placebo (n=21, 22, 24, 19 respectively) and ENTPD1, CCL13, CD79B and MRC1 expression≥median in Vigil® (n=23, 24, 26, 21, respectively) and placebo (n=23, 22, 20, 25, respectively) treated patients. P values are one sided.

FIGS. 5A-5F illustrate stratification of patient population by homologous recombination and p53 mutation status. KM curves of ENTPD1≥median expression in Vigil® (n=11) versus placebo (n=9) patients for OS (a) and RFS (b) in the HRP population. HRP, p53 mutant with high ENTPD1 expression KM curves in Vigil® (n=7) versus placebo (n=6) are presented for OS (c) and RFS (d). KM curves of HRD patients with high ENTPD1 expression in Vigil® (n=12) versus placebo (n=14) for OS (e) and RFS (f). P values are one sided.

FIG. 6 is a schematic showing the bi-shRNA^{furin} (SEQ ID NO:2) comprising two stem-loop structures each with a miR-30a loop; the first stem-loop structure has complete complementary guiding strand and passenger strand, while the second stem-loop structure has three basepair (bp) mismatches at positions 9 to 11 of the passenger strand.

### DETAILED DESCRIPTION OF THE DISCLOSURE

### I. Definitions

Unless defined otherwise, all technical and scientific terms used herein have the same meaning as is commonly understood by one of skill in the art to which the claimed subject matter belongs.

The terms “administration” and “administering”, as used herein, refer to the delivery of a bioactive composition such as a transfected tumor cell or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

As used herein, ranges and amounts can be expressed as “about” a particular value or range. About also includes the exact amount. Hence “about 5 μg” means “about 5 μg” and also “5 μg.” Generally, the term “about” includes an amount that would be expected to be within experimental error. In some embodiments, “about” refers to the number or value recited, “+” or “−” 20%, 10%, or 5% of the number or value.

The term “cancer,” as used herein, refers to the presence of cells possessing several characteristics typical of cancer-causing cells, such as uncontrolled proliferation, immortality, metastatic potential, rapid growth and proliferation rate, and certain characteristic morphological features. Some types of cancer cells can aggregate into a mass, such as a tumor, but some cancer cells can exist alone within a subject. A tumor can be a solid tumor, a non-solid tumor, a soft tissue tumor, or a metastatic lesion. Non-limiting examples of cancer include ovarian, breast, melanoma and lung. Cancer can include premalignant, as well as malignant cancers.

The terms “effective amount” or “therapeutically effective amount,” as used herein, refer to a sufficient amount of an agent or a compound being administered which will relieve to some extent one or more of the symptoms of the disease or condition being treated or prevent the onset or recurrence of the one or more symptoms of the disease or condition being treated. In some embodiments, the result is reduction and/or alleviation of the signs, symptoms, or causes of a disease, or any other desired alteration of a biological system. For example, an “effective amount” for therapeutic uses is the amount of the expression vector or autologous cancer cell vaccine required to provide a clinically significant decrease in disease symptoms without undue adverse side effects. In another example, an “effective amount” for therapeutic uses is the amount of the expression vector or autologous cancer cell vaccine as disclosed herein required to prevent a relapse of disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, is an amount effective to achieve a desired effect or therapeutic improvement without undue adverse side effects. It is understood that, in some embodiments, “an effective amount” or “a therapeutically effective amount” varies from subject to subject, due to variation in metabolism of the expression vector or autologous cancer cell vaccine, age, weight, general condition of the subject, the condition being treated, the severity of the condition being treated, and the judgment of the prescribing physician.

The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed.

As used herein, the terms “subject,” “individual,” and “patient” are used interchangeably. None of the terms are to be interpreted as requiring the supervision of a medical professional (e.g., a doctor, nurse, physician's assistant, orderly, hospice worker). As used herein, the subject is any animal, including mammals (e.g., a human or non-human animal) and non-mammals. In one embodiment of the methods and autologous tumor cell vaccines provided herein, the mammal is a human.

As used herein, the terms “treat,” “treating,” or “treatment,” and other grammatical equivalents, including, but not limited to, alleviating, abating, or ameliorating one or more symptoms of a disease or condition, ameliorating, preventing or reducing the appearance, severity, or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, preventing relapse of the disease or condition, or inhibiting the symptoms of the disease or condition either prophylactically and/or therapeutically. In a non-limiting example, for prophylactic benefit, an expression vector or autologous cancer cell vaccine composition disclosed herein is administered to an individual at risk of developing a particular disease or condition, predisposed to developing a particular disease or condition, or to an individual previously suffering from and treated for the disease or condition.

A clinical outcome can be defined using different endpoints. The term “long-term” survival is used herein to refer to survival for a particular time period, e.g., for at least 3 years, more preferably for at least 5 years. The term “Recurrence-Free Survival” (RFS) is used herein to refer to survival for a time period (usually in months or years) from randomization to first cancer recurrence or death due to recurrence of cancer. The term “Overall Survival” (OS) is used herein to refer to the time (in months or years) from randomization to death from any cause. The term “Disease-Free Survival” (DFS) is used herein to refer to survival for a time period (usually in months or years) from randomization to first cancer recurrence or death from any cause.

The terms “correlated” and “associated” are used interchangeably herein to refer to a strength of association between two measurements (or measured entities). The disclosure provides genes and gene subsets, the expression levels of which are associated with a particular outcome measure. For example, the increased expression level of a gene may be positively correlated (positively associated) with an increased likelihood of good clinical outcome for the patient, such as an increased likelihood of long-term survival without recurrence of the cancer and/or metastasis-free survival. Such a positive correlation may be demonstrated statistically in various ways, e.g. by a low hazard ratio (e.g. HR<1.0). In another example, the increased expression level of a gene may be negatively correlated (negatively associated) with an increased likelihood of good clinical outcome for the patient. In that case, for example, the patient may have a decreased likelihood of long-term survival without recurrence of the cancer and/or cancer metastasis, and the like. Such a negative correlation indicates that the patient likely has a poor prognosis, e.g., a high hazard ratio (e.g., HR>1.0).

As used herein, the term “responsiveness” or “response” refers to a positive reaction or change of a disease towards a therapy, e.g., a cancer's positive reaction towards a cancer therapy. A cancer's responsiveness to a cancer therapy can be measured by assessing the appearance, severity, and/or frequency of the symptoms of the cancer. In some embodiments, a cancer's responsiveness to a cancer therapy can be measured by the cancer patient's overall survival or relapse-free survival.

As used herein, the gene “ENTPD1” is Ectonucleoside triphosphate diphosphohydrolase-1 (gene: ENTPD1; protein: NTPDase1, HGNC: 3363) also known as CD39 (Cluster of Differentiation 39), is a typical cell surface enzyme with a catalytic site on the extracellular face.

As used herein, the gene “CCL13” is chemokine (C—C motif) ligand 13 (CCL13), which is a small cytokine belonging to the CC chemokine family (HGNC: 10611). Its gene is located on human chromosome 17 within a large cluster of other CC chemokines. CCL13 induces chemotaxis in monocytes, eosinophils, T lymphocytes, and basophils by binding cell surface G-protein linked chemokine receptors such as CCR2, CCR3 and CCR5. Activity of this chemokine has been implicated in allergic reactions such as asthma. CCL13 can be induced by the inflammatory cytokines interleukin-1 and TNF-α.

As used herein, the gene “CD79B” is immunoglobulin-associated beta, also known as CD79B (Cluster of Differentiation 79B), which is a human gene identified as HGNC: 1699.

As used herein, the gene “MRC1” is the mannose receptor C-type 1, identified as HGNC: 7228. The recognition of complex carbohydrate structures on glycoproteins is an important part of several biological processes, including cell-cell recognition, serum glycoprotein turnover, and neutralization of pathogens. The protein encoded by this gene is a type I membrane receptor that mediates the endocytosis of glycoproteins by macrophages.

As used herein, the terms “likely to” or “increased likelihood,” refer to an increased probability that an item, object, thing or individual will occur. Thus, in one example, an individual that is likely to respond to treatment with Vigil, alone or in combination with another therapy (e.g., checkpoint inhibitor), has an increased probability of responding to treatment of Vigil® alone or in combination, relative to a reference individual or group of individuals. “Unlikely to” refers to a decreased probability that an event, item, object, thing or individual will occur with respect to a reference. Thus, an individual that is unlikely to respond to treatment with Vigil, alone or in combination with another therapy, has a decreased probability of responding alone or in combination, relative to a reference individual or group of individuals.

As used herein, the phrase profiling the expression level of a gene means measuring the gene expression of the gene. Gene expression can be determined using a variety of techniques. One skilled in the art will appreciate that the expression level of a gene generally refers to “a determined level” of gene expression. This may be a determined level of gene expression as an absolute value or compared to a reference gene (e.g. a housekeeping gene), to the average of two or more reference genes, or to a computed average expression value (e.g., in DNA chip analysis) or to another informative gene without the use of a reference sample. The expression level of a gene may be measured directly, e.g., by obtaining a signal wherein the signal strength is correlated to the amount of mRNA transcripts of that gene or it may be obtained indirectly at a protein level, e.g., by immunohistochemistry, flow cytometry, CISH, ELISA or RIA methods. The expression level may also be obtained by way of a competitive reaction to a reference sample. An expression value which is determined by measuring some physical parameter in an assay, e.g. fluorescence emission, may be assigned a numerical value which may be used for further processing of information. The profiling expression levels of a gene includes one or more nucleic-acid-based analytical assays such as, for example, single-cell sequencing, single sample gene set enrichment analysis, northern blotting, fluorescent in-situ hybridization (FISH), polymerase chain reaction (PCR), real-time PCR, reverse transcription polymerase chain reaction (RT-PCR), quantitative reverse transcription PCR (qRT-PCR), serial analysis of gene expression (SAGE), microarray, or tiling arrays.

As used herein, the term “transfection” refers to the introduction of foreign DNA into eukaryotic cells. In some embodiments, transfection is accomplished by any suitable means, such as for example, calcium phosphate-DNA co-precipitation, DEAE-dextran-mediated transfection, polybrene-mediated transfection, electroporation, microinjection, liposome fusion, lipofection, protoplast fusion, retroviral infection, or biolistics.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. In some embodiments, nucleic acid molecules are composed of monomers that are naturally-occurring nucleotides (such as DNA and RNA), or analogs of naturally-occurring nucleotides (e.g., α-enantiomeric forms of naturally-occurring nucleotides), or a combination of both. In some embodiments, modified nucleotides have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, in some embodiments, the entire sugar moiety is replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. In some embodiments, nucleic acid monomers are linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. In some embodiments, the term “nucleic acid” or “nucleic acid molecule” also includes so-called “peptide nucleic acids,” which comprise naturally-occurring or modified nucleic acid bases attached to a polyamide backbone. In some embodiments, nucleic acids are single stranded or double stranded.

As used herein, the term “expression vector” refers to nucleic acid molecules encoding a gene that is expressed in a host cell. In some embodiments, an expression vector comprises a transcription promoter, a gene, and a transcription terminator. In some embodiments, gene expression is placed under the control of a promoter, and such a gene is said to be “operably linked to” the promoter. In some embodiments, a regulatory element and a core promoter are operably linked if the regulatory element modulates the activity of the core promoter. As used herein, the term “promoter” refers to any DNA sequence which, when associated with a structural gene in a host yeast cell, increases, for that structural gene, one or more of 1) transcription, 2) translation, or 3) mRNA stability, compared to transcription, translation or mRNA stability (longer half-life of mRNA) in the absence of the promoter sequence, under appropriate growth conditions.

As used herein the term “bi-functional” refers to a shRNA having two mechanistic pathways of action, that of the siRNA and that of the miRNA. The term “traditional” shRNA refers to a DNA transcription derived RNA acting by the siRNA mechanism of action. The term “doublet” shRNA refers to two shRNAs, each acting against the expression of two different genes but in the “traditional” siRNA mode.

As used herein, the term “homologous recombination deficiency-positive,” “HRD-positive,” and “HRD” are used interchangeably and they refer to the status that HR is deficient. Conversely, the term “homologous recombination deficiency-negative,” “HRD-negative,” “homologous recombination proficient,” and “HRP” are used interchangeably, and they refer to the status that HR is not deficient.

As used herein, if a gene name is followed by “wt,” it means that the genotype of the gene is wild-type.

As used herein, if a gene name is followed by “m,” it means that the genotype of the gene is mutated.

Cox regression (or Cox proportional hazards regression) is a statistical method to analyze the effect of several risk factors on survival, or in general on the time it takes for a specific event to happen. The probability of the endpoint (death, or any other event of interest, e.g. recurrence of disease) is called the hazard. The hazard is modeled as:

$$H ( t ) = {H}_{0} ( t ) \times exp ( {b}_{1} {X}_{1} + {b}_{2} {X}_{2} + {b}_{3} {X}_{3} + ⋯ + {b}_{k} {X}_{k} )$$

where X₁ . . . . Xₖ are a collection of predictor variables and H₀(t) is the baseline hazard at time t, representing the hazard for a person with the value 0 for all the predictor variables.
By dividing both sides of the above equation by H₀(t) and taking logarithms, the following is obtained:

$$ln ( \frac{H ( t )}{{H}_{0} ( t )} ) = {b}_{1} {X}_{1} + {b}_{2} {X}_{2} + {b}_{3} {X}_{3} + ⋯ + {b}_{k} {X}_{k}$$

Wherein H(t)/H₀(t) the hazard ratio. The coefficients bᵢ . . . bₖ are estimated by Cox regression and can be interpreted in a similar manner to that of multiple logistic regression.

### II. Methods of Treating Cancer

Vigil® is an autologous tumor DNA immunotherapy transfected with a plasmid encoding GM-CSF and bifunctional short hairpin RNA inhibitor against furin. Furin is an enzyme essential for cleaving TGF-beta into its active form. Vigil® was designed to enhance the immune system's potency against cancer in 3 ways: first, Vigil® introduces the individual tumor neoantigen repertoire to the immune system; second, Vigil® enhances differentiation and activation of immune cells via GM-CSF, a cytokine important to immune activation at both the peripheral and marrow levels; and finally, Vigil® inhibits cancer expressing TGF-beta, thereby decreasing immunosuppressive activity of TGF-beta. Functional immune activation of Vigil® in correlation with clinical benefit has been demonstrated via ELISPOT assay. Moreover, Vigil® appears to increase CD3+/CD8+ T cell circulation in advanced solid tumor patients and expands MHC-II expression activity via NanoString analysis in correlation with clinical benefit. Safety and efficacy of Vigil® has been evaluated in several tumor types in addition to ovarian cancer.

A randomized double-blind placebo-controlled study (VITAL trial) of Vigil® versus placebo as maintenance therapy for frontline Stage III/IV ovarian cancer recently demonstrated clinical benefit from randomization in recurrence free survival (RFS) and overall survival (OS) in patients with BRCAwt tumors. The disclosure describes molecular analysis of biomarker profiles that best identify the patient subpopulations most sensitive to Vigil® therapy. The disclosure identifies high ENTPD1 gene expression level is highly correlated to Vigil® therapy sensitivity all patient populations.

In one embodiment, the disclosure provides a method for treating an individual having cancer, the method comprising:
profiling an expression level of one or more genes selected from the group consisting of ENTPD1, CCL13, CD79B, and MRC1, in a tumor obtained from said individual;
determining the presence of an elevated expression level of the one or more genes selected from the group consisting of ENTPD1, CCL13, CD79B, and MRC1 in the tumor obtained from said individual; and
administering to the individual a therapy comprising an expression vector having (i) a first insert comprising a nucleic acid sequence encoding a Granulocyte Macrophage Colony Stimulating Factor (GM-CSF) sequence; and (ii) a second insert,
wherein the second insert encodes one or more bifunctional short hairpin RNAs (shRNA) capable of hybridizing to one of more regions of a mRNA transcript encoding furin, wherein at least one of the regions is selected from base sequences 300-318, 731-740, 1967-1991, 2425-2444, 2827-2851 or 2834-2852 of SEQ ID NO:1, thereby inhibiting furin expression via RNA interference, wherein each bifunctional short hairpin RNA comprises a first stem-loop structure that comprises an siRNA component and a second stem-loop structure that comprises a miRNA component and wherein the shRNA incorporates siRNA (cleavage dependent) and miRNA (cleavage-independent) motifs, and wherein the therapy improves the treatment response in the individual with the elevated expression level compared to a patient with a tumor having a low expression level of the gene.

In some embodiments of this aspect, the second insert comprises two stem-loop structures each with a miR-30a loop, and the first stem-loop structure has complete complementary guiding strand and passenger strand, while the second stem-loop structure has three basepair (bp) mismatches at positions 9 to 11 of the passenger strand. In particular embodiments, the second insert comprises a sequence having at least 90% (e.g., 91%, 92%, 93%, 94%, 95%, 96%, 97%, 98%, 99%, or 100%) identity to the sequence of SEQ ID NO:2. In certain embodiments, the second insert comprises the sequence of SEQ ID NO:2.

In another embodiment, the disclosure provides a method for predicting responsiveness of an individual having or suspected of having cancer to a therapy comprising an expression vector having (i) a first insert comprising a nucleic acid sequence encoding a Granulocyte Macrophage Colony Stimulating Factor (GM-CSF) sequence; and (ii) a second insert, the method comprising:
profiling the expression level of one or more genes selected from the group consisting of ENTPD1, CCL13, CD79B, and MRC1, in a tumor obtained from said individual;
determining the presence of an elevated expression level of the one or more genes selected from the group consisting of ENTPD1, CCL13, CD79B, and MRC1; and
identifying the individual with the elevated expression level of the one or more genes as predicted to have an increased responsiveness to the therapy, compared to a patient with a tumor having a low expression of the gene,
wherein the second insert encodes one or more bifunctional short hairpin RNAs (shRNA) capable of hybridizing to one of more regions of a mRNA transcript encoding furin, wherein at least one of the regions is selected from base sequences 300-318, 731-740, 1967-1991, 2425-2444, 2827-2851 or 2834-2852 of SEQ ID NO:1, thereby inhibiting furin expression via RNA interference, wherein each bifunctional short hairpin RNA comprises a first stem-loop structure that comprises an siRNA component and a second stem-loop structure that comprises a miRNA component and wherein the shRNA incorporates siRNA (cleavage dependent) and miRNA (cleavage-independent) motifs.

In some embodiments of this aspect, the second insert comprises two stem-loop structures each with a miR-30a loop, and the first stem-loop structure has complete complementary guiding strand and passenger strand, while the second stem-loop structure has three basepair (bp) mismatches at positions 9 to 11 of the passenger strand. In particular embodiments, the second insert comprises a sequence having at least 90% (e.g., 91%, 92%, 93%, 94%, 95%, 96%, 97%, 98%, 99%, or 100%) identity to the sequence of SEQ ID NO:2. In certain embodiments, the second insert comprises the sequence of SEQ ID NO:2.

In still another embodiment, the disclosure provides A method for selecting an individual having cancer to be subjected to a therapy comprising an expression vector having (i) a first insert comprising a nucleic acid sequence encoding a Granulocyte Macrophage Colony Stimulating Factor (GM-CSF) sequence; and (ii) a second insert, the method comprising:
profiling the expression level of one or more genes selected from the group consisting of ENTPD1, CCL13, CD79B, and MRC1, in a tumor obtained from said individual;
determining the presence of an elevated expression level of the one or more genes selected from the group consisting of ENTPD1, CCL13, CD79B, and MRC1; and
selecting the individual who is determined to have the elevated expression level of the one or more genes compared to a patient with a tumor having a low expression of the gene as an individual to be subjected to the therapy,
wherein the second insert encodes one or more bifunctional short hairpin RNAs (shRNA) capable of hybridizing to one of more regions of a mRNA transcript encoding furin, wherein at least one of the regions is selected from base sequences 300-318, 731-740, 1967-1991, 2425-2444, 2827-2851 or 2834-2852 of SEQ ID NO:1, thereby inhibiting furin expression via RNA interference, wherein each bifunctional short hairpin RNA comprises a first stem-loop structure that comprises an siRNA component and a second stem-loop structure that comprises a miRNA component and wherein the shRNA incorporates siRNA (cleavage dependent) and miRNA (cleavage-independent) motifs.

In some embodiments of this aspect, the second insert comprises two stem-loop structures each with a miR-30a loop, and the first stem-loop structure has complete complementary guiding strand and passenger strand, while the second stem-loop structure has three basepair (bp) mismatches at positions 9 to 11 of the passenger strand. In particular embodiments, the second insert comprises a sequence having at least 90% (e.g., 91%, 92%, 93%, 94%, 95%, 96%, 97%, 98%, 99%, or 100%) identity to the sequence of SEQ ID NO:2. In certain embodiments, the second insert comprises the sequence of SEQ ID NO:2.

The disclosure provides methods for predicting responsiveness in a subject to a cancer treatment, comprising determining the expression level of an ENTPD1 gene in a sample from the subject, wherein the cancer treatment comprises administering to the subject an expression vector comprising: (a) a first insert comprising a nucleic acid sequence encoding a Granulocyte Macrophage Colony Stimulating Factor (GM-CSF) sequence; and (b) a second insert comprising a sequence having at least 90% (e.g., 91%, 92%, 93%, 94%, 95%, 96%, 97%, 98%, 99%, or 100%) identity to the sequence of SEQ ID NO:2 (e.g., SEQ ID NO: 2); and wherein a determination of at least 1.1-fold (e.g., at least 2-fold, 3-fold, 4-fold, 5-fold, 6-fold, 7-fold, 8-fold, 9-fold, or 10-fold) higher expression level of the ENTPD1 gene in the sample from the subject than the expression level of the ENTPD1 gene in a control sample (e.g., a control sample selected from the group consisting of a healthy subject, a cancer subject, and a previously treated cancer subject) indicates that the subject is responsive to the cancer treatment.

In some embodiments, in addition to determining the expression level of ENTPD1, the methods include determining the expression level of other biomarker genes, which include for example, one or more of CCL13, CD79B, and MRC1.

In some embodiments, in addition to determining the expression level of ENTPD1 gene, the methods further comprise the determination of the genotypes of at least two genes selected from the group consisting of BRCA1, BRCA2, and TP53. In some embodiments, the methods further comprise determining the genotypes of BRCA1 and BRCA2. In some embodiments, the methods further comprise determining the genotypes of BRCA1 and TP53. In some embodiments, the methods further comprise determining the genotypes of BRCA2 and TP53. In some embodiments, the methods further comprise determining the genotypes of BRCA1 and BRCA2. In some embodiments, the methods further comprise determining the genotypes of BRCA1, BRCA2, and TP53.

Certain genotypes of BRCA1, BRCA2, and/or TP53, in addition to a higher expression level of the ENTPD1 gene, indicate that the subject is likely to respond to the cancer treatment (e.g., Vigil® therapy). In some embodiments, in addition to a higher expression level of the ENTPD1 gene, a determination of BRCA1wt and BRCA2wt indicates that the subject is likely to respond to the cancer treatment (e.g., Vigil® therapy). In some embodiments, in addition to a higher expression level of the ENTPD1 gene, a determination of TP53m and BRCA1wt indicates that the subject is likely to respond to the cancer treatment (e.g., Vigil® therapy). In some embodiments, in addition to a higher expression level of the ENTPD1 gene, a determination of TP53m and BRCA2wt indicates that the subject is likely to respond to the cancer treatment (e.g., Vigil® therapy). In some embodiments, in addition to a higher expression level of the ENTPD1 gene, a determination of BRCA1wt and BRCA2wt and TP53m indicates that the subject is likely to respond to the cancer treatment (e.g., Vigil® therapy).

In some embodiments of the methods described herein, the subject can be homologous recombination deficiency (HRD)-negative or HRD-positive. In particular embodiments, the subject is more likely to be responsive to the cancer treatment (e.g., Vigil® therapy) if the subject is HRD-negative.

In further embodiments, the methods described herein, upon the determination of the expression level of the ENTPD1 gene that indicates responsiveness of the subject to the cancer treatment, further comprise treating the subject with the cancer treatment (e.g., Vigil® therapy).

The disclosure also provides methods for predicting the responsiveness of a cancer in a subject to a cancer treatment, comprising: a) measuring the expression level of the ENTPD1 gene in a sample from the subject to determine at least 1.1-fold (e.g., at least 2-fold, 3-fold, 4-fold, 5-fold, 6-fold, 7-fold, 8-fold, 9-fold, or 10-fold) higher expression level of the ENTPD1 gene in the sample from the subject than the expression level of the ENTPD1 gene in a control sample (e.g., a control sample selected from the group consisting of a healthy subject, a cancer subject, and a previously treated cancer subject); b) administering to the subject an expression vector comprising: i) a first insert comprising a nucleic acid sequence encoding a Granulocyte Macrophage Colony Stimulating Factor (GM-CSF) sequence; and ii) a second insert comprising a sequence according to SEQ ID NO:2, to thereby treat the subject.

In some embodiments, the subject receiving the cancer treatment may be identified as homologous recombination proficient (e.g., HRD-negative). In some embodiments, the subject receiving the cancer treatment has the genotypes BRCA1wt, BRCA2wt, and/or TP53m (e.g., BRCA1wt and BRCA2wt, BRCA1wt and TP53m, BRCA2wt and TP53m, and BRCA1wt and BRCA2wt and TP53m).

In some embodiments, one or more available sequencing techniques can be used to determine the genotype of one or more genes in the subject. In some embodiments, the sequencing comprises Sanger sequencing or next generation sequencing. In some embodiments, the next generation sequencing comprises massively parallel sequencing. In some embodiments, determining the genotypes comprises hybridization of nucleic acid extracted from the individual to an array. In some embodiments, the array is a microarray. In some embodiments, determining the genotypes comprises array comparative genomic hybridization of nucleic acid extracted from the individual.

In the methods described herein, in some embodiments, a sample can be a tissue sample. In some embodiments, a sample can be a biopsy sample from the patient, such as a biopsy sample of the tumor cells or a biopsy sample of circulating tumor cells.

In some embodiments, to characterize whether an individual is HRD-positive or HRD-negative, an HRD score can be determined. In some embodiments, an HRD score can be calculated based on scores for the loss of heterozygosity (LOH), telomeric allelic imbalance (TAI), and large-scale state transitions (LSTs). In some embodiments, the LOH is indicated by the presence of a single allele. In some embodiments, the LOH is defined as the number of chromosomal loss of heterozygosity regions longer than 15 Mb. In some embodiments, the TAI is indicated by a discrepancy in the 1 to 1 allele ratio at the end of the chromosome. In some embodiments, the LSTs are indicated by transition points between regions of abnormal and normal DNA or between two different regions of abnormality. In some embodiments, the LSTs are defined as the number of break points between regions longer than 10 Mb after filtering out regions shorter than 3 Mb. In certain embodiments, the HRD score is calculated as the sum of the LOH, TAI, and LST scores. Methods of determining an HRD score is available in the art, e.g., as described in Takaya et al., Sci Rep. 10 (1): 2757, 2020, Telli et al., Clin Cancer Res 22 (15): 3764-73, 2016, and Marchetti and McNeish, Cancer Breaking News 5 (1): 15-20, 2017. Further, commercial services for HRD score determination are also available, for example, services provided by Ambry Genetics, Caris Life Sciences, Counsylgenetic, Foundation Medicine, GeneDX, Integrated Genetics, Invitae, Myriad Genetics, and Neogenomics. In some embodiments, an individual having the genotype BRCA1wt, BRCA2wt, and/or TP53m (e.g., BRCA1wt and BRCA2wt, BRCA1wt and TP53m, BRCA2wt and TP53m, and BRCA1wt and BRCA2wt and TP53m) can be HRD-negative or HRD-positive. In some embodiments, an individual having the genotype BRCA1wt, BRCA2wt, and/or TP53m (e.g., BRCA1wt and BRCA2wt, BRCA1wt and TP53m, BRCA2wt and TP53m, and BRCA1wt and BRCA2wt and TP53m) is HRD-negative. In particular embodiments, an individual identified as having an HRD-positive status has an HRD score of 42 or greater (e.g., 42, 43, 44, 45, 46, 47, 48, 49, 50, 51, 52, 53, 54, 55, 56, 57, 58, 59, 60, or greater).

One skilled in the art will recognize that there are statistical methods that may be used to determine whether there is a significant relationship between an outcome of interest (e.g., likelihood of survival, likelihood of response to therapy) and expression levels of a marker gene as described herein. This relationship can be presented as a continuous recurrence score (RS), or patients may be stratified into risk groups (e.g., low, intermediate, high). For example, a Cox proportional hazards regression model may fit to a particular clinical endpoint (e.g., RFS, DFS, OS). One assumption of the Cox proportional hazards regression model is the proportional hazards assumption, i.e., the assumption that effect parameters multiply the underlying hazard.

### III. Nanostring Analysis

As described above, in an effort to identify the patient subpopulation most sensitive to Vigil® therapy based on molecular profile, NanoString® assessment was used.

### Step 1: Initial Gene Selection Using Univariate Analysis in Vigil® Patients Only

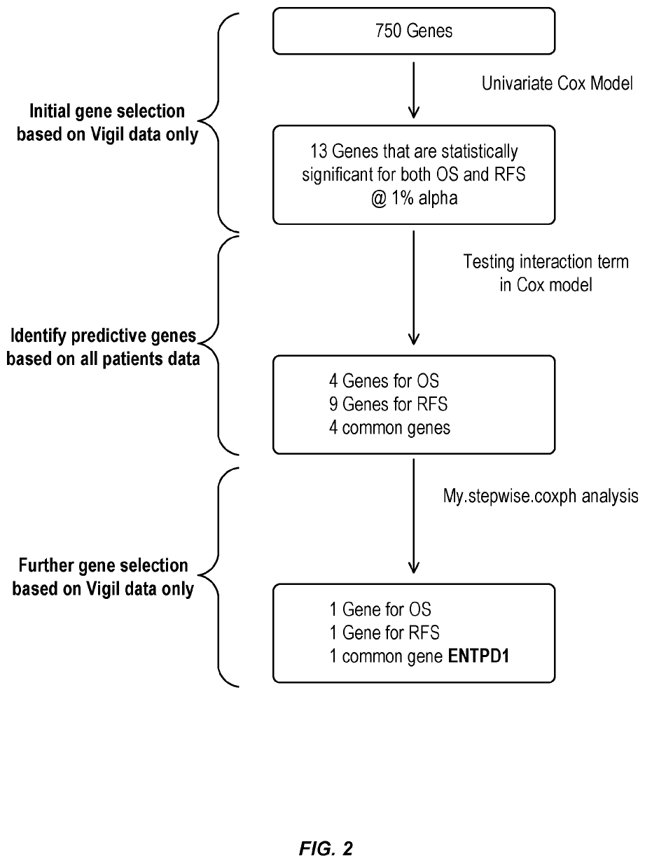

As is shown in FIG. 2, for each of the 750 genes in the NanoString platform, a univariate Cox model (Step 1) was used with gene Z-scores (i.e., gene data from NanoString) as a continuous variable and run for both OS and RFS in Vigil® treated patients. From this data, the p-value, HR and corresponding 95% confidence interval (CI) were extracted. Genes that were significant for both OS and RFS advantage at the 1% significance level were identified. The more stringent variable selection criterion of 1% significance level was used due to the relatively small number of OS/RFS events compared to the large number of genes assessed. When such algorithm is applied on new data, the 1% significance level can be adjusted to other thresholds (e.g., 2%, 5%, etc.) depending on number of OS/RFS events compared to the number of genes assessed. The Z score as referenced above is a measure of distance in standard deviations of a sample from the mean. If the Z score is negative, expression is positively correlated with a good prognosis; if positive, expression is negatively correlated to a good prognosis.

### Step 2: Identify Predictive Genes Using all Patients Data

For each of the genes identified in Step 1, further analyses were performed using all patients' data (including Vigil® patients and Placebo patients) to determine if it is predictive of Vigil® treatment advantage. The Cox proportional hazards model with interaction term for each gene identified in Step 1 was used to identify genes that were predictive of response to Vigil® by analyzing data of both Vigil® and placebo patients. A Cox proportional hazards model was used to determine if the interaction term between gene and treatment group was significant. The Cox model included the treatment group, gene and treatment-by gene interaction term. The gene was considered predictive if the interaction term was significant (p≤0.05). The 5% level of significance can be adjusted depending on real data. The model was run using the gene as a continuous variable or using binary high or low gene assignment. When using binary gene assignment, the median gene value for all 91 patients was calculated for each of the 750 cancer expression pathway genes. Patients were dichotomized into high or low gene expression groups if their value was either above or below the median. Other thresholds instead of median gene values can be used for dichotomization depending on the scientific rationale and research question. Kaplan-Meier (KM) curves were generated for genes identified as predictive for both OS and RFS.

### Step 3: Further Gene Selection Using Multivariate Analysis in Vigil® Patients Only

Since the identified predictive genes in Step 2 may not be independent, further model selection was performed using a univariant Cox model in Vigil® treated patients to further refine identification of relevant genes. We used the my.stepwise.coxph function in R (open source, R Core Team), which employs both forward selection and backward elimination methodology to further select genes that were significantly associated with the time-to-event data (OS or RFS) in Vigil® treated patient. The significance level for variable entry and for stay in the model was set at 0.01 and variable stay we set at 0.01 to account for potential multiplicity in the model selection process. The best candidate final multi-variate Cox model in Vigil® treated patients was identified manually by dropping the covariates with p value>0.01 one at a time until all regression coefficients were significantly different from 0 at an alpha level of 0.01. When such algorithm is applied on new data, the 0.01 significance level can be adjusted to other thresholds (e.g., 1%, 2%, 5%, etc.) depending on number of OS/RFS events compared to the number of genes assessed.

Once genes are identified as above, gene expression can be determined using a variety of techniques. One skilled in the art will appreciate that the expression level of a gene generally refers to “a determined level” of gene expression. This may be a determined level of gene expression as an absolute value or compared to a reference gene (e.g. a housekeeping gene), to the average of two or more reference genes, or to a computed average expression value (e.g., in DNA chip analysis) or to another informative gene without the use of a reference sample. The expression level of a gene may be measured directly, e.g., by obtaining a signal wherein the signal strength is correlated to the amount of mRNA transcripts of that gene or it may be obtained indirectly at a protein level, e.g., by immunohistochemistry, flow cytometry, CISH, ELISA or RIA methods. The expression level may also be obtained by way of a competitive reaction to a reference sample. An expression value which is determined by measuring some physical parameter in an assay, e.g. fluorescence emission, may be assigned a numerical value which may be used for further processing of information.

In some embodiments, the profiling expression levels of a gene includes one or more nucleic-acid-based analytical assays such as, for example, single-cell sequencing, single sample gene set enrichment analysis, northern blotting, fluorescent in-situ hybridization (FISH), polymerase chain reaction (PCR), real-time PCR, reverse transcription polymerase chain reaction (RT-PCR), quantitative reverse transcription PCR (qRT-PCR), serial analysis of gene expression (SAGE), microarray, or tiling arrays.

### IV. Expression Vector

In some embodiments, the Granulocyte Macrophage Colony Stimulating Factor (GM-CSF) in the expression vector is a human GM-CSF sequence. In some embodiments, the expression vector further comprises a promoter, e.g., the promoter is a cytomegalovirus (CMV) mammalian promoter. In some embodiments, the mammalian CMV promoter comprises a CMV immediate early (IE) 5′ UTR enhancer sequence and a CMV IE Intron A. In further embodiments, the expression vector further comprises a CMV enhancer sequence and a CMV intron sequence.

The first insert and the second insert in the expression vector can be operably linked to the promoter. In particular embodiments, the expression vector further comprises a nucleic acid sequence encoding a picornaviral 2A ribosomal skip peptide between the first and the second nucleic acid inserts.

In some embodiments, the expression vector comprises at least one bifunctional shRNA (bi-shRNA). In some embodiments, the bi-shRNA comprises a first stem-loop structure that comprises an siRNA component and a second stem-loop structure that comprises a miRNA component. In some embodiments, the bi-functional shRNA has two mechanistic pathways of action, that of the siRNA and that of the miRNA. Thus, in some embodiments, the bi-functional shRNA described herein is different from a traditional shRNA, i.e., a DNA transcription derived RNA acting by the siRNA mechanism of action or from a “doublet shRNA” that refers to two shRNAs, each acting against the expression of two different genes but in the traditional siRNA mode. In some embodiments, the bi-shRNA incorporates siRNA (cleavage dependent) and miRNA (cleavage-independent) motifs.

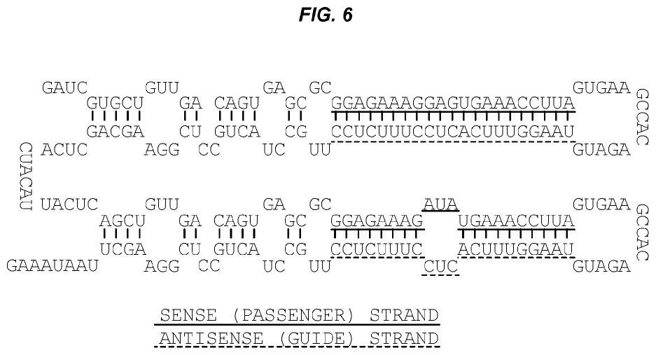

In some embodiments, the at least one bi-shRNA is capable of hybridizing to one of more regions of an mRNA transcript encoding furin. In some embodiments, the mRNA transcript encoding furin is a nucleic acid sequence of SEQ ID NO:1. In some embodiments, the one or more regions of the mRNA transcript encoding furin is selected from base sequences 300-318, 731-740, 1967-1991, 2425-2444, 2827-2851, and 2834-2852 of SEQ ID NO: 1. In some embodiments, the expression vector targets the coding region of the furin mRNA transcript, the 3′ UTR region sequence of the furin mRNA transcript, or both the coding sequence and the 3′ UTR sequence of the furin mRNA transcript simultaneously. In some embodiments, the bi-shRNA comprises SEQ ID NO:2. In some embodiments, a bi-shRNA capable of hybridizing to one or more regions of an mRNA transcript encoding furin is referred to herein as bi-shRNA^{furin}. In some embodiments, the bi-shRNA^{furin} comprises or consists of two stem-loop structures each with miR-30a backbone. In some embodiments, a first stem-loop structure of the two stem-loop structures comprises complementary guiding strand and passenger strand (FIG. 6). In some embodiments, the second stem-loop structure of the two stem-loop structures comprises three mismatches in the passenger strand. In some embodiments, the three mismatches are at positions 9 to 11 in the passenger strand.

The expression vector can comprise: a. a first insert comprising a nucleic acid sequence encoding a Granulocyte Macrophage Colony Stimulating Factor (GM-CSF) sequence; and b. a second insert comprising two stem-loop structures each with a miR-30a loop; the first stem-loop structure has complete complementary guiding strand and passenger strand, while the second stem-loop structure has three base pair (bp) mismatches at positions 9 to 11 of the passenger strand. Descriptions of the miR-30a loop and its sequence are known in the art, see, e.g., Rao et al., Cancer Gene Ther. 17 (11): 780-91, 2010; Jay et al., Cancer Gene Ther. 20 (12): 683-9, 2013; Rao et al., Mol Ther. 24 (8): 1412-22, 2016; Phadke et al., DNA Cell Biol. 30 (9): 715-26, 2011; Barve et al., Mol Ther. 23 (6): 1123-1130, 2015; Rao et al., Methods Mol Biol. 942:259-78, 2013; and Senzer et al., Mol Ther. 20 (3): 679-86, 2012. In some embodiments, the miR-30a loop comprises the sequence of GUGAAGCCACAGAUG (SEQ ID NO:6). In some embodiments, the guiding strand in the first stem-loop structure comprises the sequence of SEQ ID NO:4 and the passenger strand in the first stem-loop structure has the sequence of SEQ ID NO:3. In some embodiments, the guiding strand in the second stem-loop structure comprises the sequence of SEQ ID NO:4 and the passenger strand in the second stem-loop structure has the sequence of SEQ ID NO:5.

TABLE A
Sequences

| SEQ ID | | |
|---|---|---|
| NO | Description | Sequence |
| 1 | mRNA | GCGGGGAAGCAGCAGCGGCCAGGATGAATCCCAGGT |
| | transcript | GCTCTGGAGCTGGATGGTGAAGGTCGGCACTCTTCAC |
| | encoding | CCTCCCGAGCCCTGCCCGTCTCGGCCCCATGCCCCCAC |
| | furin | CAGTCAGCCCCGGGCCACAGGCAGTGAGCAGGCACCT |
| | | GGGAGCCGAGGCCCTGTGACCAGGCCAAGGAGACGG |
| | | GCGCTCCAGGGTCCCAGCCACCTGTCCCCCCCATGGA |
| | | GCTGAGGCCCTGGTTGCTATGGGTGGTAGCAGCAACA |
| | | GGAACCTTGGTCCTGCTAGCAGCTGATGCTCAGGGCC |
| | | AGAAGGTCTTCACCAACACGTGGGCTGTGCGCATCCC |
| | | TGGAGGCCCAGCGGTGGCCAACAGTGTGGCACGGAAG |
| | | CATGGGTTCCTCAACCTGGGCCAGATCTTCGGGGACT |
| | | ATTACCACTTCTGGCATCGAGGAGTGACGAAGCGGTC |
| | | CCTGTCGCCTCACCGCCCGCGGCACAGCCGGCTGCAG |
| | | AGGGAGCCTCAAGTACAGTGGCTGGAACAGCAGGTGG |
| | | CAAAGCGACGGACTAAACGGGACGTGTACCAGGAGC |
| | | CCACAGACCCCAAGTTTCCTCAGCAGTGGTACCTGTCT |
| | | GGTGTCACTCAGCGGGACCTGAATGTGAAGGCGGCCT |
| | | GGGCGCAGGGCTACACAGGGCACGGCATTGTGGTCTC |
| | | CATTCTGGACGATGGCATCGAGAAGAACCACCCGGAC |
| | | TTGGCAGGCAATTATGATCCTGGGGCCAGTTTTGATGT |
| | | CAATGACCAGGACCCTGACCCCCAGCCTCGGTACACA |
| | | CAGATGAATGACAACAGGCACGGCACACGGTGTGCGG |
| | | GGGAAGTGGCTGCGGTGGCCAACAACGGTGTCTGTGG |
| | | TGTAGGTGTGGCCTACAACGCCCGCATTGGAGGGGTG |
| | | CGCATGCTGGATGGCGAGGTGACAGATGCAGTGGAGG |
| | | CACGCTCGCTGGGCCTGAACCCCAACCACATCCACAT |
| | | CTACAGTGCCAGCTGGGGCCCCGAGGATGACGGCAAG |
| | | ACAGTGGATGGGCCAGCCCGCCTCGCCGAGGAGGCCT |
| | | TCTTCCGTGGGGTTAGCCAGGGCCGAGGGGGGCTGGG |
| | | CTCCATCTTTGTCTGGGCCTCGGGGAACGGGGGCCGG |
| | | GAACATGACAGCTGCAACTGCGACGGCTACACCAACA |
| | | GTATCTACACGCTGTCCATCAGCAGCGCCACGCAGTTT |
| | | GGCAACGTGCCGTGGTACAGCGAGGCCTGCTCGTCCA |
| | | CACTGGCCACGACCTACAGCAGTGGCAACCAGAATGA |
| | | GAAGCAGATCGTGACGACTGACTTGCGGCAGAAGTGC |
| | | ACGGAGTCTCACACGGGCACCTCAGCCTCTGCCCCCTT |
| | | AGCAGCCGGCATCATTGCTCTCACCCTGGAGGCCAAT |
| | | AAGAACCTCACATGGCGGGACATGCAACACCTGGTGG |
| | | TACAGACCTCGAAGCCAGCCCACCTCAATGCCAACGA |
| | | CTGGGCCACCAATGGTGTGGGCCGGAAAGTGAGCCAC |
| | | TCATATGGCTACGGGCTTTTGGACGCAGGCGCCATGG |
| | | TGGCCCTGGCCCAGAATTGGACCACAGTGGCCCCCCA |
| | | GCGGAAGTGCATCATCGACATCCTCACCGAGCCCAAA |
| | | GACATCGGGAAACGGCTCGAGGTGCGGAAGACCGTG |
| | | ACCGCGTGCCTGGGCGAGCCCAACCACATCACTCGGC |
| | | TGGAGCACGCTCAGGCGCGGCTCACCCTGTCCTATAA |
| | | TCGCCGTGGCGACCTGGCCATCCACCTGGTCAGCCCC |
| | | ATGGGCACCCGCTCCACCCTGCTGGCAGCCAGGCCAC |
| | | ATGACTACTCCGCAGATGGGTTTAATGACTGGGCCTTC |
| | | ATGACAACTCATTCCTGGGATGAGGATCCCTCTGGCG |
| | | AGTGGGTCCTAGAGATTGAAAACACCAGCGAAGCCAA |
| | | CAACTATGGGACGCTGACCAAGTTCACCCTCGTACTCT |
| | | ATGGCACCGCCCCTGAGGGGCTGCCCGTACCTCCAGA |
| | | AAGCAGTGGCTGCAAGACCCTCACGTCCAGTCAGGCC |
| | | TGTGTGGTGTGCGAGGAAGGCTTCTCCCTGCACCAGA |
| | | AGAGCTGTGTCCAGCACTGCCCTCCAGGGTTCGCCCCC |
| | | CAAGTCCTCGATACGCACTATAGCACCGAGAATGACG |
| | | TGGAGACCATCCGGGCCAGCGTCTGCGCCCCCTGCCA |
| | | CGCCTCATGTGCCACATGCCAGGGGCCGGCCCTGACA |
| | | GACTGCCTCAGCTGCCCCAGCCACGCCTCCTTGGACCC |
| | | TGTGGAGCAGACTTGCTCCCGGCAAAGCCAGAGCAGC |
| | | CGAGAGTCCCCGCCACAGCAGCAGCCACCTCGGCTGC |
| | | CCCCGGAGGTGGAGGCGGGGCAACGGCTGCGGGCAG |
| | | GGCTGCTGCCCTCACACCTGCCTGAGGTGGTGGCCGG |
| | | CCTCAGCTGCGCCTTCATCGTGCTGGTCTTCGTCACTG |
| | | TCTTCCTGGTCCTGCAGCTGCGCTCTGGCTTTAGTTTTC |
| | | GGGGGGTGAAGGTGTACACCATGGACCGTGGCCTCAT |
| | | CTCCTACAAGGGGCTGCCCCCTGAAGCCTGGCAGGAG |
| | | GAGTGCCCGTCTGACTCAGAAGAGGACGAGGGCCGGG |
| | | GCGAGAGGACCGCCTTTATCAAAGACCAGAGCGCCCT |
| | | CTGATGAGCCCACTGCCCACCCCCTCAAGCCAATCCCC |
| | | TCCTTGGGCACTTTTTAATTCACCAAAGTATTTTTTTAT |
| | | CTTGGGACTGGGTTTGGACCCCAGCTGGGAGGCAAGA |
| | | GGGGTGGAGACTGCTTCCCATCCTACCCTCGGGCCCA |
| | | CCTGGCCACCTGAGGTGGGCCCAGGACCAGCTGGGGC |
| | | GTGGGGAGGGCCGTACCCCACCCTCAGCACCCCTTCC |
| | | ATGTGGAGAAAGGAGTGAAACCTTTAGGGCAGCTTGC |
| | | CCCGGCCCCGGCCCCAGCCAGAGTTCCTGCGGAGTGA |
| | | AGAGGGGCAGCCCTTGCTTGTTGGGATTCCTGACCCA |
| | | GGCCGCAGCTCTTGCCCTTCCCTGTCCCTCTAAAGCAA |
| | | TAATGGTCCCATCCAGGCAGTCGGGGGCTGGCCTAGG |
| | | AGATATCTGAGGGAGGAGGCCACCTCTCCAAGGGCTT |
| | | CTGCACCCTCCACCCTGTCCCCCAGCTCTGGTGAGTCT |
| | | TGGCGGCAGCAGCCATCATAGGAAGGGACCAAGGCA |
| | | AGGCAGGTGCCTCCAGGTGTGCACGTGGCATGTGGCC |
| | | TGTGGCCTGTGTCCCATGACCCACCCCTGTGCTCCGTG |
| | | CCTCCACCACCACTGGCCACCAGGCTGGCGCAGCCAA |
| | | GGCCGAAGCTCTGGCTGAACCCTGTGCTGGTGTCCTG |
| | | ACCACCCTCCCCTCTCTTGCACCCGCCTCTCCCGTCAG |
| | | GGCCCAAGTCCCTGTTTTCTGAGCCCGGGCTGCCTGGG |
| | | CTGTTGGCACTCACAGACCTGGAGCCCCTGGGTGGGT |
| | | GGTGGGGAGGGGCGCTGGCCCAGCCGGCCTCTCTGGC |
| | | CTCCCACCCGATGCTGCTTTCCCCTGTGGGGATCTCAG |
| | | GGGCTGTTTGAGGATATATTTTCACTTTGTGATTATTT |
| | | CACTTTAGATGCTGATGATTTGTTTTTGTATTTTTAATG |
| | | GGGGTAGCAGCTGGACTACCCACGTTCTCACACCCAC |
| | | CGTCCGCCCTGCTCCTCCCTGGCTGCCCTGGCCCTGAG |
| | | GTGTGGGGGCTGCAGCATGTTGCTGAGGAGTGAGGAA |
| | | TAGTTGAGCCCCAAGTCCTGAAGAGGCGGGCCAGCCA |
| | | GGCGGGCTCAAGGAAAGGGGGTCCCAGTGGGAGGGG |
| | | CAGGCTGACATCTGTGTTTCAAGTGGGGCTCGCCATGC |
| | | CGGGGGTTCATAGGTCACTGGCTCTCCAAGTGCCAGA |
| | | GGTGGGCAGGTGGTGGCACTGAGCCCCCCCAACACTG |
| | | TGCCCTGGTGGAGAAAGCACTGACCTGTCATGCCCCC |
| | | CTCAAACCTCCTCTTCTGACGTGCCTTTTGCACCCCTC |
| | | CCATTAGGACAATCAGTCCCCTCCCATCTGGGAGTCCC |
| | | CTTTTCTTTTCTACCCTAGCCATTCCTGGTACCCAGCCA |
| | | TCTGCCCAGGGGTGCCCCCTCCTCTCCCATCCCCCTGC |
| | | CCTCGTGGCCAGCCCGGCTGGTTTTGTAAGATGCTGGG |
| | | TTGGTGCACAGTGATTTTTTTCTTGTAATTTAAACAGG |
| | | CCCAGCATTGCTGGTTCTATTTAATGGACATGAGATAA |
| | | TGTTAGAGGTTTTAAAGTGATTAAACGTGCAGACTAT |
| | | GCAAACCAG |
| 2 | bi-shRNA^{furin} | GAUCCUGCUGUUGACAGUGAGCGCGGAGAAAGGAGU |
| | | GAAACCUUAGUGAAGCCACAGAUGUAAGGUUUCACU |
| | | CCUUUCUCCUUGCCUACUGCCUCGGAAGCAGCUCAC |
| | | UACAUUACUCAGCUGUUGACAGUGAGCGCGGAGAAA |
| | | GAUAUGAAACCUUAGUGAAGCCACAGAUGUAAGGUU |
| | | UCACUCCUUUCUCCUUGCCUACUGCCUCGGAAGCUU |
| | | AAUAAAGGAUCUUUUAUUUUCAUUGGAUC |
| 3 | Passenger | GGAGAAAGGAGUGAAACCUUA |
| | strand in | |
| | first | |
| | stem-loop | |
| | structure in | |
| | bi-shRNA^{furin} | |
| 4 | Guide strand | UAAGGUUUCACUCCUUUCUCC |
| | in both first | |
| | and second | |
| | stem-loop | |
| | structures in | |
| | bi-shRNA^{furin} | |
| 5 | Passenger | GGAGAAAGAUAUGAAACCUUA |
| | strand in | |
| | second stem- | |
| | loop | |
| | structure in | |
| | bi-shRNA^{furin} | |
| 6 | miR-30a | GUGAAGCCACAGAUG |
| | loop | |

In some embodiments, the expression vector plasmid can have a sequence that is at least 90% (e.g., 90%, 91%, 92%, 93%, 94%, 95%, 96%, 97%, 98%, 99%, or 100%) identical to the sequence of SEQ ID NO:7. The vector plasmid can comprise a first nucleic acid insert operably linked to a promoter, wherein the first insert encodes the GM-CSF cDNA, a second nucleic acid insert operably linked to the promoter, wherein the second insert encodes one or more short hairpin RNAs (shRNA) capable of hybridizing to a region of a mRNA transcript encoding furin, thereby inhibiting furin expression via RNA interference.

SEQ ID NO: 7:
GGCCATTGCATACGTTGTATCCATATCATAATATGTACATTTATATTGGCTCATGT
CCAACATTACCGCCATGTTGACATTGATTATTGACTAGTTATTAATAGTAATCAA
TTACGGGGTCATTAGTTCATAGCCCATATATGGAGTTCCGCGTTACATAACTTAC
GGTAAATGGCCCGCCTGGCTGACCGCCCAACGACCCCCGCCCATTGACGTCAAT
AATGACGTATGTTCCCATAGTAACGCCAATAGGGACTTTCCATTGACGTCAATGG
GTGGAGTATTTACGGTAAACTGCCCACTTGGCAGTACATCAAGTGTATCATATGC
CAAGTACGCCCCCTATTGACGTCAATGACGGTAAATGGCCCGCCTGGCATTATGC
CCAGTACATGACCTTATGGGACTTTCCTACTTGGCAGTACATCTACGTATTAGTC
ATCGCTATTACCATGGTGATGCGGTTTTGGCAGTACATCAATGGGCGTGGATAGC
GGTTTGACTCACGGGGATTTCCAAGTCTCCACCCCATTGACGTCAATGGGAGTTT
GTTTTGGCACCAAAATCAACGGGACTTTCCAAAATGTCGTAACAACTCCGCCCCA
TTGACGCAAATGGGCGGTAGGCGTGTACGGTGGGAGGTCTATATAAGCAGAGCT
CGTTTAGTGAACCGTCAGATCGCCTGGAGACGCCATCCACGCTGTTTTGACCTCC
ATAGAAGACACCGGGACCGATCCAGCCTCCGCGGCCGGGAACGGTGCATTGGAA
CGCGGATTCCCCGTGCCAAGAGTGACGTAAGTACCGCCTATAGACTCTATAGGC
ACACCCCTTTGGCTCTTATGCATGCTATACTGTTTTTGGCTTGGGGCCTATACACC
CCCGCTTCCTTATGCTATAGGTGATGGTATAGCTTAGCCTATAGGTGTGGGTTATT
GACCATTATTGACCACTCCAACGGTGGAGGGCAGTGTAGTCTGAGCAGTACTCGT
TGCTGCCGCGCGCGCCACCAGACATAATAGCTGACAGACTAACAGACTGTTCCTT
TCCATGGGTCTTTTCTGCAGTCACCGTCGTCGACGGTATCGATAAGCTTGATATC
GAATTCCGCTGGAGGATGTGGCTGCAGAGCCTGCTGCTCTTGGGCACTGTGGCCT
GCAGCATCTCTGCACCCGCCCGCTCGCCCAGCCCCAGCACGCAGCCCTGGGAGC
ATGTGAATGCCATCCAGGAGGCCCGGCGTCTCCTGAACCTGAGTAGAGACACTG
CTGCTGAGATGAATGAAACAGTAGAAGTCATCTCAGAAATGTTTGACCTCCAGG
AGCCGACCTGCCTACAGACCCGCCTGGAGCTGTACAAGCAGGGCCTGCGGGGCA
GCCTCACCAAGCTCAAGGGCCCCTTGACCATGATGGCCAGCCACTACAAGCAGC
ACTGCCCTCCAACCCCGGAAACTTCCTGTGCAACCCAGACTATCACCTTTGAAAG
TTTCAAAGAGAACCTGAAGGACTTTCTGCTTGTCATCCCCTTTGACTGCTGGGAG
CCAGTCCAGGAGTGAGACCGGCCAGATGAGGCTGGCCAAGCCGGGGAGCTGCTC
TCTCATGAAACAAGAGCTAGAAACTCAGGATGGTCATCTTGGAGGGACCAAGGG
GTGGGCCACAGCCATGGTGGGAGTGGCCTGGACCTGCCCTGGGCCACACTGACC
CTGATACAGGCATGGCAGAAGAATGGGAATATTTTATACTGACAGAAATCAGTA
ATATTTATATATTTATATTTTTAAAATATTTATTTATTTATTTATTTAAGTTCATAT
TCCATATTTATTCAAGATGTTTTACCGTAATAATTATTATTAAAAATATGCTTCTA
AAAAAAAAAAAAAAAAAAAAAACGGAATTCACGTGGGCCCGGTACCGTATACT
CTAGAAGATCTGGCAGCGGAGAGGGCAGAGGAAGTCTTCTAACATGCGGTGACG
TGGAGGAGAATCCCGGCCCTAGGATGTCTAGAGCGGCCGCGGATCCTGCTGTTG
ACAGTGAGCGCGGAGAAAGGAGTGAAACCTTAGTGAAGCCACAGATGTAAGGTT
TCACTCCTTTCTCCTTGCCTACTGCCTCGGAAGCAGCTCACTACATTACTCAGCTG
TTGACAGTGAGCGCGGAGAAAGATATGAAACCTTAGTGAAGCCACAGATGTAAG
GTTTCACTCCTTTCTCCTTGCCTACTGCCTCGGAAGCTTAATAAAGGATCTTTTAT
TTTCATTGGATCCAGATCTTTTTCCCTCTGCCAAAAATTATGGGGACATCATGAA
GCCCCTTGAGCATCTGACTTCTGGCTAATAAAGGAAATTTATTTTCATTGCAATA
GTGTGTTGGAATTTTTTGTGTCTCTCACTCGGAAGGACATATGGGAGGGCAAATC
ATTTAAAACATCAGAATGAGTATTTGGTTTAGAGTTTGGCAACATATGCCCATTC
TTCCGCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAG
TCCAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGCCT
AACTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAAGGATC
TCAAGAAGATCCTTTGATCTTTTCTACGGGGTCTGACGCTCAGTGGAACGAAAAC
TCACGTTAAGGGATTTTGGTCATGAGATTATCAAAAAGGATCTTCACCTAGATCC
TTTTAAATTAAAAATGAAGTTTTAAATCAATCTAAAGTATATATGAGTAAACTTG
GTCTGACAGTTACCAATGCTTAATCAGTGAGGCACCTATCTCAGCGATCTGTCTA
TTTCGTTCATCCATAGTTGCCTGACTCGGGGGGGGGGGGCGCTGAGGTCTGCCTC
GTGAAGAAGGTGTTGCTGACTCATACCAGGCCTGAATCGCCCCATCATCCAGCCA
GAAAGTGAGGGAGCCACGGTTGATGAGAGCTTTGTTGTAGGTGGACCAGTTGGT
GATTTTGAACTTTTGCTTTGCCACGGAACGGTCTGCGTTGTCGGGAAGATGCGTG
ATCTGATCCTTCAACTCAGCAAAAGTTCGATTTATTCAACAAAGCCGCCGTCCCG
TCAAGTCAGCGTAATGCTCTGCCAGTGTTACAACCAATTAACCAATTCTGATTAG
AAAAACTCATCGAGCATCAAATGAAACTGCAATTTATTCATATCAGGATTATCAA
TACCATATTTTTGAAAAAGCCGTTTCTGTAATGAAGGAGAAAACTCACCGAGGC
AGTTCCATAGGATGGCAAGATCCTGGTATCGGTCTGCGATTCCGACTCGTCCAAC
ATCAATACAACCTATTAATTTCCCCTCGTCAAAAATAAGGTTATCAAGTGAGAAA
TCACCATGAGTGACGACTGAATCCGGTGAGAATGGCAAAAGCTTATGCATTTCTT
TCCAGACTTGTTCAACAGGCCAGCCATTACGCTCGTCATCAAAATCACTCGCATC
AACCAAACCGTTATTCATTCGTGATTGCGCCTGAGCGAGACGAAATACGCGATC
GCTGTTAAAAGGACAATTACAAACAGGAATCGAATGCAACCGGCGCAGGAACAC
TGCCAGCGCATCAACAATATTTTCACCTGAATCAGGATATTCTTCTAATACCTGG
AATGCTGTTTTCCCGGGGATCGCAGTGGTGAGTAACCATGCATCATCAGGAGTAC
GGATAAAATGCTTGATGGTCGGAAGAGGCATAAATTCCGTCAGCCAGTTTAGTCT
GACCATCTCATCTGTAACATCATTGGCAACGCTACCTTTGCCATGTTTCAGAAAC
AACTCTGGCGCATCGGGCTTCCCATACAATCGATAGATTGTCGCACCTGATTGCC
CGACATTATCGCGAGCCCATTTATACCCATATAAATCAGCATCCATGTTGGAATT
TAATCGCGGCCTCGAGCAAGACGTTTCCCGTTGAATATGGCTCATAACACCCCTT
GTATTACTGTTTATGTAAGCAGACAGTTTTATTGTTCATGATGATATATTTTTATC
TTGTGCAATGTAACATCAGAGATTTTGAGACACAACGTGGCTTTCCCCCCCCCCC
CATTATTGAAGCATTTATCAGGGTTATTGTCTCATGAGCGGATACATATTTGAAT
GTATTTAGAAAAATAAACAAATAGGGGTTCCGCGCACATTTCCCCGAAAAGTGC
CACCTGACGTCTAAGAAACCATTATTATCATGACATTAACCTATAAAAATAGGCG
TATCACGAGGCCCTTTCGTCTCGCGCGTTTCGGTGATGACGGTGAAAACCTCTGA
CACATGCAGCTCCCGGAGACGGTCACAGCTTGTCTGTAAGCGGATGCCGGGAGC
AGACAAGCCCGTCAGGGCGCGTCAGCGGGTGTTGGCGGGTGTCGGGGCTGGCTT
AACTATGCGGCATCAGAGCAGATTGTACTGAGAGTGCACCATATGCGGTGTGAA
ATACCGCACAGATGCGTAAGGAGAAAATACCGCATCAGATTGGCTATT

An expression vector comprising a first nucleic acid encoding GM-CSF and a second nucleic acid encoding at least one bifunctional short hairpin RNA (bi-shRNA) capable of hybridizing to a region of an mRNA transcript encoding furin is referred to as a bishRNA^{furin}/GMCSF expression vector.

### V. Cancer Cells

In some embodiments, the expression vectors used in methods described herein are within autologous cancer cells, e.g., autologous tumor cells, xenograft expanded autologous tumor cells, allogeneic tumor cells, xenograft expanded allogeneic tumor cells, or combinations thereof. In some embodiments, the autologous cancer cell is transfected with the expression vector. In some embodiments, the cells are autologous tumor cells. In some embodiments, the allogenic tumor cells are established cell lines. In some embodiments, autologous tumor cells are obtained from the individual in need thereof. In some embodiments, when the cells are autologous tumor cells, the composition is referred to as an autologous tumor cell vaccine. In some embodiments, the autologous tumor cell vaccine comprises from 1×10⁶ cells to about 5×10⁷ cells, such as 1×10⁶ cells, 2×10⁶ cells, 3×10⁶ cells, 4×10⁶ cells, 5×10⁶ cells, 6×10⁶ cells, 7×10⁶ cells, 8×10⁶ cells, 9×10⁶ cells, 1×10⁷ cells, 2×10⁷ cells, 3×10⁷ cells, 4×10⁷ cells, or 5×10⁷ cells.

In some embodiments, the cells are harvested from an individual. In some embodiments, the cells are harvested from a tissue of the individual. In some embodiments, the tissue is a tumor tissue. In some embodiments, the tumor tissue is ovarian tumor tissue. In some embodiments, the tumor tissue is harvested during a biopsy or a cytoreduction surgery on the individual. In some embodiments, the tumor tissue or cells from the tumor tissue are placed in an antibiotic solution in a sterile container. In some embodiments, the antibiotic solution comprises gentamicin, sodium chloride, or a combination thereof.

### VI. Cancers and Survival

In some embodiments, the cancer is an HRD-negative, wild-type BRCA1/2 cancer. In some embodiments, the cancer is selected from the group consisting of a solid tumor cancer, ovarian cancer, adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, prostate cancer, sarcoma, stomach cancer, uterine cancer, thyroid cancer, and a hematological cancer. Examples of solid tumor cancers include, but are not limited to, endometrial cancer, biliary cancer, bladder cancer, liver hepatocellular carcinoma, gastric/esophageal cancer, ovarian cancer, melanoma, breast cancer, pancreatic cancer, colorectal cancer, glioma, non-small-cell lung carcinoma, prostate cancer, cervical cancer, kidney cancer, thyroid cancer, a neuroendocrine cancer, small cell lung cancer, a sarcoma, head and neck cancer, brain cancer, clear cell renal cell carcinoma, skin cancer, endocrine tumor, thyroid cancer, tumor of unknown origin, and a gastrointestinal stromal tumor.

In particular embodiments of the methods, the cancer is ovarian cancer. In some embodiments, the method can prevent or delay relapse of a substantially eradicated ovarian cancer. The substantially eradicated ovarian cancer can be Stage III or Stage IV ovarian cancer. In other embodiments, the cancer can be breast cancer, melanoma, or lung cancer. In some embodiments, Stage III ovarian cancer means that the cancer is found in one or both ovaries and has spread outside the pelvis to other parts of the abdomen and/or nearby lymph nodes. It is also considered Stage III ovarian cancer when it has spread to the surface of the liver. In Stage IV ovarian cancer, the cancer has spread beyond the abdomen to other parts of the body, such as the lungs or tissue inside the liver. Cancer cells in the fluid around the lungs is also considered Stage IV ovarian cancer.

In certain embodiments, the ovarian cancer is Stage III or Stage IV ovarian cancer. In some embodiments, the Stage III ovarian cancer is Stage IIIb or worse. In some embodiments, the ovarian cancer is a high-grade serous ovarian carcinoma, a clear cell ovarian carcinoma, endometroid ovarian carcinoma, mucinous ovarian carcinoma, or a low-grade serous ovarian carcinoma.

In some embodiments of the methods, a relapse free survival (RFS) of the individual is increased relative to an individual with substantially eradicated ovarian cancer who has not been administered the expression vector or autologous tumor cell vaccine containing the expression vector.

As used herein, the term “relapse free survival” refers to the time after administration of an initial therapy to treat a cancer that the cancer remains undetectable (i.e., until the cancer relapses). In some embodiments, relapse free survival of an individual receiving the expression vector or the autologous cancer cell vaccine containing the expression vector is from 5 months to 11 months longer than relapse free survival of an individual not receiving the expression vector or the autologous cancer cell vaccine containing the expression vector. In some embodiments, relapse free survival of an individual receiving the expression vector or the autologous cancer cell vaccine containing the expression vector is at least 5 months, 6 months, 7 months 8 months, 9 months, 10 months, or 11 months longer than relapse free survival of an individual not receiving the expression vector or the autologous cancer cell vaccine containing the expression vector.

As used herein, the term “substantially eradicated” refers to an ovarian cancer which is not detectable in an individual following an initial therapy to treat the ovarian cancer. In some embodiments, detection of ovarian cancer, or lack thereof, is by a chest x-ray, computed tomography (CT) scan, magnetic resonance imaging (MRI), detection of a cancer antigen 125 (CA-125) level, physical examination or presence of symptoms suggestive of active cancer, or any combination thereof. In some embodiments, a detection of cancer antigen 125 (CA-125) levels of ≤35 units/ml indicates no ovarian cancer is present in the individual. In some embodiments, an ovarian cancer which has been substantially eradicated can be referred to as having achieved a clinical complete response (cCR).

In some embodiments, relapse free survival of an individual receiving the expression vector or the autologous cancer cell vaccine containing the expression vector is at least 5 months longer than relapse free survival of an individual not receiving the expression vector or the autologous cancer cell vaccine containing the expression vector. In some embodiments, relapse free survival of a BRCAwt individual receiving the expression vector or the autologous cancer cell vaccine containing the expression vector is greater than 15 months from time of surgical debulking, wherein a relapse free survival of an individual not receiving the expression vector or the autologous cancer cell vaccine containing the expression vector is less than 15 months from time of surgical debulking. In some embodiments, relapse free survival of a BRCAwt individual receiving the expression vector or the autologous cancer cell vaccine containing the expression vector is at least 11 months longer than relapse free survival of an individual not receiving the expression vector or the autologous cancer cell vaccine containing the expression vector.

In some embodiments, the individual received an initial therapy. In some embodiments, administration of an initial therapy results in a clinical completely response of the cancer to the therapy. In some embodiments, the initial therapy comprises debulking, administration of a chemotherapy, administration of a therapeutic agent, or the combination thereof. In some embodiments, the chemotherapy comprises a platinum-based drug, a taxane, or a combination thereof. In some embodiments, the platinum-based drug comprises cisplatin, carboplatin, oxaliplatin, nedaplatin, triplatin tetranitrate, phenanthriplatin, picoplatin, satraplatin, or a combination thereof. In some embodiments, the platinum-based drug comprises carboplatin. In some embodiments, the taxane comprises paclitaxel, docetaxel, cabazitaxel, or a combination thereof. In some embodiments, the taxane comprises paclitaxel. In some embodiments, the therapeutic agent comprises an angiogenesis inhibitor, a PARP inhibitor, a checkpoint inhibitor, or a combination thereof. In some embodiments, the angiogenesis inhibitor comprises a vascular endothelial growth factor (VEGF) inhibitor. In some embodiments, the VEGF inhibitor comprises sorafenib, sunitinib, bevacizumab, pazopanib, axitinib, cabozantinib, levatinib, or a combination thereof. In some embodiments, the VEGF inhibitor is bevacizumab. In some embodiments, the PARP inhibitor comprises olaparib, rucaparib, niraparib, talazoparib, veliparib, pamiparib, or a combination thereof. In some embodiments, the checkpoint inhibitor comprises a PD-1 inhibitor, a PD-L1 inhibitor, a CTLA-4 inhibitor, or a combination thereof. In some embodiments, the checkpoint inhibitor comprises pembrolizumab, nivolumab, cemiplimab, atezolizumab, avelumab, durvalumab, ipilimumab, or a combination thereof. In some embodiments, the ovarian cancer is resistant or refractory to the chemotherapy or the therapeutic agent.

### VII. Administration, Formulations, and Dosing

In some embodiments, the autologous cancer cell vaccine containing the expression vector comprises about 1×10⁶ or about 1×10⁷ autologous cancer cells transfected as described herein. In some embodiments, the autologous cancer cell vaccine comprises at least 1×10⁶ or at least 1×10⁷ autologous cancer cells transfected as described herein. In some embodiments, the autologous cancer cell vaccine comprises from about 1×10⁶ cells to about 1×10⁷ (e.g., 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, or 1×10⁷) autologous cancer cells transfected as described herein. In some embodiments, the autologous cancer cell vaccine comprises from about 1×10⁶ cells to about 2.5×10⁷ (e.g., 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 1×10⁷, 1.5×10⁷, 2×10⁷, or 2.5×10⁷) autologous cancer cells transfected as described herein. In some embodiments, the autologous cancer cell vaccine comprises from about 1×10⁶ cells to about 5×10⁷ (e.g., 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, or 5×10⁷) autologous cancer cells transfected as described herein.

In some embodiments, the autologous cancer cell vaccine further comprises one or more vaccine adjuvants.

In some embodiments, the expression vector or the autologous cancer cell vaccine is in a unit dosage form. The term “unit dosage form”, as used herein, describes a physically discrete unit containing a predetermined quantity of the expression vector or the autologous cancer cell vaccine described herein, in association with other ingredients (e.g., vaccine adjuvants). In some embodiments, the predetermined quantity is a number of cells.

In some embodiments, an individual is administered one dose of the expression vector or the autologous cancer cell vaccine per month. In some embodiments, a dose of the expression vector or the autologous cancer cell vaccine is administered to the individual once a month for from 1 months to 12 months. In some embodiments, the individual is administered at least one dose of the expression vector or the autologous cancer cell vaccine. In some embodiments, the individual is administered no more than twelve doses of the expression vector or the autologous cancer cell vaccine. In some embodiments, the individual is administered 1, 2, 3, 4, 5, 6, 7, 8, 9, 10, 11, or 12 doses of the expression vector or the autologous cancer cell vaccine. In some embodiments, the dose is a unit dosage form of the expression vector or the autologous cancer cell vaccine. In some embodiments, a dose of the expression vector or the autologous cancer cell vaccine is administered to the individual every three months, every two months, once a month, twice a month, or three times a month. In some embodiments, the expression vector or the autologous cancer cell vaccine is administered to the individual for up to 1 month, 2 months, 3 months, 4 months, 5 months, 6 months, 12 months, 18 months, 24 months, or 36 months. In some embodiments, the expression vector or the autologous cancer cell vaccine is administered to the individual by injection. In some embodiments, the injection is an intradermal injection. In some embodiments, a first dose of the expression vector or the autologous cancer cell vaccine is administered to the individual following confirmation of the individual achieving a clinical complete response (cCR). In some embodiments, a first dose of the expression vector or the autologous cancer cell vaccine is administered to the individual no earlier than the same day as the final treatment of the initial therapy. In some embodiments, a first dose of the expression vector or the autologous cancer cell vaccine is administered to the individual no later than 8 weeks following the final treatment of the initial therapy.

### VIII. Combinations

In some embodiments, the expression vector or the autologous cancer cell vaccine is administered with an additional therapeutic agent. In some embodiments, the additional therapeutic agent comprises a therapeutically effective dose of γIFN (gamma interferon). In some embodiments, the therapeutically effective dose of γIFN is from about 50 μg/m² to about 100 μg/m². In some embodiments, the therapeutically effective dose of γIFN is about 50 μg/m², about 60 μg/m², about 70 μg/m², about 80 μg/m², about 90 μg/m², or about 100 μg/m². In some embodiments, the additional therapeutic agent comprises an angiogenesis inhibitor, a PARP inhibitor, a checkpoint inhibitor, or a combination thereof. In some embodiments, the angiogenesis inhibitor comprises a vascular endothelial growth factor (VEGF) inhibitor. In some embodiments, the VEGF inhibitor comprises sorafenib, sunitinib, bevacizumab, pazopanib, axitinib, cabozantinib, levatinib, or a combination thereof. In some embodiments, the VEGF inhibitor is bevacizumab. In some embodiments, the PARP inhibitor comprises olaparib, rucaparib, niraparib, talazoparib, veliparib, pamiparib, or a combination thereof. In some embodiments, the checkpoint inhibitor comprises a PD-1 inhibitor, a PD-L1 inhibitor, a CTLA-4 inhibitor, or a combination thereof. In some embodiments, the checkpoint inhibitor comprises pembrolizumab, nivolumab, cemiplimab, atezolizumab, avelumab, durvalumab, ipilimumab, or a combination thereof.

Using NanoString PanCancer Immuno-Oncology 360™ molecular profiles derived from patient tumor samples in conjunction with NanoString Statistical Algorithm (NSA), it was determined that high expression of ENTPD1/CD39 was associated with a significant and independent improvement in OS and RFS with Vigil® maintenance therapy in the VITAL study. ENTPD1/CD39 is highly expressed in OC cell-lines, and functions as a master regulator to maintain the balance between proinflammatory and immunosuppressive regulatory function. The latter largely due the role of ENTPD1/CD39 as the rate limiting step in the conversion of ATP to ADP in the adenosine pathway. Adenosine inhibits both T-cell and NK-cell anti-tumor function. Although adenosine can be exported from the tumor into the extracellular space by nucleoside transport proteins, it is primarily formed via the action of membrane ectoenzymes by phosphohydrolysis from dead cells. In addition, ENTPD1/CD39 is present on cancer extracellular vesicles (ECVs). ENTPD1/CD39 is ubiquitously expressed in the vasculature, B cells, NK cells, dendritic cells, monocytes, macrophages, regulatory T cells and monocyte derived suppressor cells in the TME. CD8+ T cells demonstrate T cell exhaustion signatures with malignant upregulation of CD39 in the tumor microenvironment. Moreover, T regulatory (Treg) cell upregulation of ENTPD1/CD39 within the tumor microenvironment generates immunosuppressive activity thereby facilitating malignant growth and survival. Inhibition of ENTPD/1CD39 in murine cancer models induces anticancer activity and ENTPD1/CD39 deficient mice demonstrated a reduction in tumor growth. Furthermore, anti-ENTPD1/CD39 increased cytotoxicity of alloreactive primed T-cell towards fresh OvCA cells.

Vigil® treated patients with baseline elevated tumor expression of ENTPD1/CD39 were associated with a significantly improved response compared to those patients with tumors with low expression and to those with high tumor expression treated with placebo. The primary VITAL study results suggest that Vigil® induction of GMCSF, knock down of TGFβ1 and TGFβ2 and induced CD8+ T cell activity targeted to tumor-specific cancer neoantigens provide anticancer activity beneficially impacts OS and RFS in newly diagnosed Stage III/IV OC patients receiving Vigil® as maintenance therapy. This activity appears to be correlated to high ENTPD1/CD39 expression—a presumptive predictive marker. Interestingly, in a murine model, high levels of TGFβ were associated with immunosuppressive CD39+ myeloid derived suppressor cells (MDSC). Notably, placebo treated patients from the VITAL study with high ENTPD1/CD39 expression tended to show poorer survival compared to patients with lower expression, presumably reflecting the immunosuppressive role of ENTPD1/CD39 in these patients. It is also of interest that ENTPD1/CD39 promotes tumor cell survival in hypoxic regions characterized by increased levels of ATP and high concentrations of vascular endothelial growth factor (VEGF), thereby supporting the consideration of a combination of Vigil® and a VEGF inhibitor in therapeutic trial.

Previously, Vigil® has shown the ability to activate a systemic immune response. In Phase IIA clinical testing, all Vigil® treated patients (n=31) demonstrated immune activation through γIFN-ELISPOT assay which correlated with durable overall survival benefit. Vigil® also demonstrated in a small number of patients increase in the number of circulating CD3+/CD8+ T cells following treatment. In the VITAL trial we demonstrated RFS and OS benefit in patients with HRP molecular profile. We also suggested that the presence of mutant p53 may further improve delineation of Vigil® responsive patients. Results of mRNA expression via NanoString signature also indicate enhanced OS and/or RFS endpoint benefits of Vigil® maintenance in both these groups. These results support the need for further verification of ENTPD1/CD39 as a biomarker of sensitivity to Vigil® treatment in OC and possibly other solid tumors with high ENTPD1/CD39 expression.

The presence of ENTPD1/CD39 in multiple cell types other than certain cancers (e.g., CD4+/Treg, CD8+ and MDSC) supports the consideration of therapeutic assessment of combined ENTPD1/CD39 inhibition and Vigil® in patients with ENTPD1/CD39^{high} tumor expression. ENTPD1/CD39 monoclonal antibodies have demonstrated anticancer activity in murine models as single agents and in combination with checkpoint inhibitors and autologous EBV-specific human T cells. Currently, there are a number of different CD39 targeting agents in early Phase I clinical trials under evaluation.

It is also possible that in a larger patient population receiving Vigil, supportive evidence demonstrated with the other immune modulatory signals identified by NanoString analysis (i.e., CXCL13, CD79B, MRC1) will also be found to have further impact on OS and RFS. All three of these genes also perform important immunologic functions. MRC1 is expressed on tumor associated macrophages (TAMs) with M2 phenotype. Once activated MRC1 directs TAM's to M1 phenotype thereby activating the innate response. Recent work has shown that high CXCL13 expression in high-grade serous ovarian cancer correlates with increased survival by maintaining CXCR5+/CD8+ T cells with in tertiary lymphoid structures. CD79b expression is limited to B cells. B cells play an important role in anti-tumor immunity through secretion of cytokines and antigen presentation. Such results may further direct research towards a multiplex of biomarker sensitivity and may even direct novel combination therapeutic approaches with Vigil, including combination treatment regimens based on various molecular signal expression patterns and immune related signal pathways that are relevant to Vigil® related benefit.

Molecular biomarker assessment to optimize the proportion of responsive patient populations to Vigil® therapy will involve more comprehensive analyses including p53mu, BRCA1/2-wt, ENTPD1/CD39 and HRP molecular profiles. Evidence provided shows these gene expression signals can act independently in defining sensitive subpopulations and also appears to support the possibility that the combined use of predictive biomarkers can suggest if not identify additive and possibly, synergistic therapeutic activity combinations. Clearly, statistical analyses such as those applied to the VITAL study here will likely continue to help identify optimal subpopulations with potential to benefit via treatment with Vigil® as well as suggest the direction for continued Vigil® combination studies. Results also justify trial consideration of Vigil® in other solid tumor patients with HRP profile, p53mu and those with ENTPD1/CD39 high expression by NanoString PanCancer Immuno-Oncology 360™ CodeSet analysis.

In conclusion, gene signatures and profiles indicative of response to Vigil® maintenance therapy were identified as part of frontline treatment in newly diagnosed OC patients. Interestingly, NSA identified ENTPD1/CD39, a gene signal associated with an immunosuppressive tumor microenvironment, was most highly predictive of Vigil® responsiveness. Previous work has indicated TGFβ may upregulate ENTPD1/CD39 in immunosuppressive myeloid cells, such that Vigil's effect in downregulating TGFβ may counter this effect and account for its activity in patients with high tumor expression of ENTPD1/CD39. Combining previously identified biomarkers of Vigil® response, such as HRP and mutant p53, with ENTPD1/CD39 expression allows for refined identification of Vigil® responsive populations-ultimately allowing a transition from predictive analysis to prescriptive analytics. Such an approach can be more broadly applied to assess for correlations between gene expression signals and survival benefits as well as widen the therapeutic index by optimizing patient selection and treatment allocation with other targeted therapies.

### EXAMPLES

### Example 1—Nanostring® Signature of Treatment Response to Gemogenovatucel-T (Vigil®) as Maintenance in Newly Diagnosed Ovarian Cancer (Vital Study)

A novel statistical algorithm was employed to identify molecular biomarkers with the strongest correlation to Vigil® treatment response to inform use in target populations most likely to respond and to direct combination therapy options for future development of Vigil®.

### Methods

### Study Design and Vigil® Construction

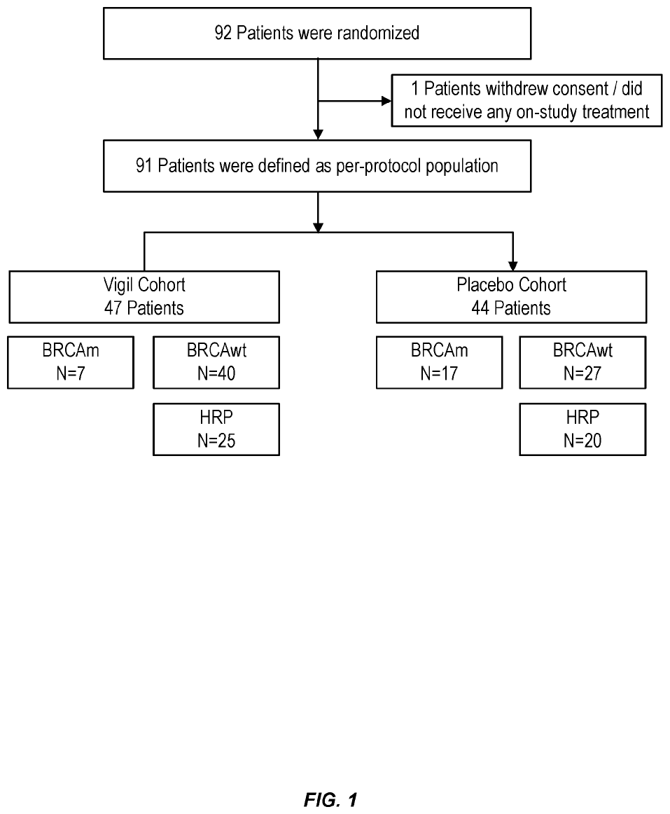

All patients provided written informed consent prior to study enrollment on the VITAL study. Briefly, the VITAL study (NCT02346747) was a phase 2b randomized, double-blind, placebo controlled trial involving women 18 years and older with stage III or IV high-grade serous, endometroid or clear cell ovarian cancer in clinical complete response. As specified in the approved clinical protocol (Mary Crowley IRB), preclinical specimens were obtained from tissue harvested at the time of procurement for vaccine manufacture. Tissue is dissociated into cell suspension and cells are frozen at a concentration of 1.33 million cells/ml in freeze media (10% DMSO v/v in 1% HSA/plasma-Lyte A solution and stored long term in vapor phase nitrogen. Homologous recombination status [homologous recombination deficient (HRD) or HRP] was determined for all patients using the Myriad MyChoice CDx assay as previously described. Patient demographics and consort diagram are presented in Table 1 and FIG. 1, respectively.

TABLE 1
Demographics summary of all patients by ENTPD1 status.

| | ENTPD1 Status, No. (%) | | |
|---|---|---|---|
| Characteristic | | ENTPD1 Low | ENTPD1 High |

| | | |
|---|---|---|
| No. of patients | 45 | 46 |

| | | | | |
|---|---|---|---|---|
| Frontline chemotherapy | | | | |
| Neoadjuvant | 6 | (13.3%) | 9 | (19.6%) |
| Adjuvant | 39 | (86.7%) | 37 | (80.4%) |
| Stage | | | | |
| III | 38 | (84.4%) | 39 | (84.8%) |
| IV | 7 | (15.6%) | 7 | (15.2%) |
| Age, years | | | | |
| Median (IQR) | 62.0 | (56-70) | 63.5 | (55-68) |

| | | |
|---|---|---|
| Range | 38-79 | 42-84 |

| | | | | |
|---|---|---|---|---|
| <65 | 27 | (60%) | 26 | (56.5%) |
| >=65 | 18 | (40%) | 20 | (43.5%) |
| ECOG | | | | |
| 0 | 31 | (68.9) | 30 | (65.2) |
| 1 | 14 | (31.1) | 16 | (34.8) |
| Residual disease post-surgery | | | | |
| Macroscopic | 13 | (28.9%) | 14 | (30.4%) |
| Microscopic/NED | 32 | (71.1%) | 32 | (69.6%) |

Vigil® plasmid construction and cGMP manufacturing have been previously described. Following VITAL study protocol guidelines, ovarian tumor tissue was excised at the time of initial tumor cytoreduction surgery and shipped to Gradalis, Inc. (Dallas, TX) for tissue processing, transfection and vaccine manufacture.

### RNA Isolation and Gene Expression Analysis

RNA expression was determined from total RNA isolated using RNeasy Mini Kit (Qiagen, Venlo, The Netherlands). NanoString PanCancer Immuno-Oncology 360™ CodeSet using the nCounter SPRINT platform (NanoString Technologies, Seattle, WA, USA), which includes 750 cancer expression pathway genes, was used to analyze gene expression per manufacturer protocol.

### Statistics

For all 750 genes a NanoString statistical algorithm (NSA) was defined prior to gene analysis (FIG. 2) to assess the correlation of NanoString gene expression results with clinical benefit as measured by both RFS and OS advantage effect with Vigil® to specific mRNA expression. First, a univariate Cox model was used with gene Z-scores as a continuous variable and run for both OS and RFS in Vigil® treated patients. From this data, the two-sided p-value, HR and corresponding 95% confidence interval (CI) were extracted. Genes that were significant for both OS and RFS advantage at the 1% significance level were identified. The more stringent variable selection criterion of 1% significance level was used due to the relatively small number of OS/RFS events compared to the large number of genes assessed. Next, Cox proportional hazards model with interaction term for each gene identified in the univariate Cox model was used to identify genes that were predictive of response to Vigil® by analyzing data of both Vigil® and placebo patients. A Cox proportional hazards model was used to determine if the interaction term between gene and treatment group was significant. The Cox model included the treatment group, gene and treatment-by-gene interaction term. The gene was considered predictive if the interaction term was significant (p≤0.05). The model was run using the gene as a continuous variable or using binary high or low gene assignment. When using binary gene assignment, the median gene value for all 91 patients was calculated for each of the 750 cancer expression pathway genes. Patients were dichotomized into high or low gene expression groups if their value was either above or below the median. Kaplan-Meier (KM) curves were generated for genes identified as predictive for both OS and RFS. Since the identified predictive genes may not be independent, further model selection was performed using a multivariate Cox model in Vigil R treated patients to further refine identification of relevant genes. We used the my.stepwise.coxph function in R (open source, R Core Team), which employs both forward selection and backward elimination methodology to further select genes that were significantly associated with the time-to-event data (OS or RFS) in Vigil® treated patients. The significance level for variable entry and for stay in the model was set at 0.01 and variable stay we set at 0.01 to account for potential multiplicity in the model selection process.

### Biomarker Vigil® Benefit Over Placebo

Previous analyses of Vigil® relationship to BRCA1/2-wt, HRP and TP53 mutation (p53mu) subpopulations revealed correlation to clinical benefit. These subpopulations were explored via KM analysis to assess the effect of combination biomarkers BRCA1/2-wt, HRP, p53mu and genes identified as significant following multivariate analysis in this study on Vigil® and placebo treatment effects as measured by OS and RFS.

### Results

### Univariate Analysis Vigil® Patients Only

First, a univariate Cox model was performed with the gene Z-score as a continuous variable to obtain the two-sided p-value, HR and 95% CI in Vigil® treated patients only (n=47). This analysis identified 13 genes that were statistically significant at the 1% significance level for both OS and RFS (Table 2). All of these genes are associated with critical immunologic modulation function as per NanoString Pan Cancer Immuno-Oncology 360™ Code set (NanoString Technologies, Seattle, WA, USA).

TABLE 2
Significant genes correlated with OS and RFS from univariate analysis

| | | HR | HR | Min | Max | p value | p value |
|---|---|---|---|---|---|---|---|
| | Locus | OS | RFS | Value | Value | OS | RFS |
| ADORA2A | NM_000675.5 | 0.099 | 0.211 | −4.22 | −2.1 | 0.0027 | 0.0006 |
| CCL13 | NM_005408.2 | 0.221 | 0.423 | −6.83 | −2.5 | 0.0034 | 0.0014 |
| CD79B | NM_001039933.1 | 0.308 | 0.447 | −6.31 | −3.01 | 0.0051 | 0.0044 |
| ENTPD1 | NM_001098175.1 | 0.243 | 0.481 | −3.99 | 0.61 | 0.0008 | 0.0006 |
| FAM124B | NM_001122779.1 | 0.134 | 0.178 | −6.24 | −4.24 | 0.0054 | 0.0006 |
| GZMB | NM_004131.4 | 0.452 | 0.561 | −6.97 | −1.02 | 0.008 | 0.0009 |
| ICOS | NM_012092.3 | 0.487 | 0.626 | −6.41 | −0.49 | 0.0088 | 0.0085 |
| IL2RA | NM_000417.2 | 0.321 | 0.478 | −5.61 | −0.74 | 0.0097 | 0.0062 |
| LAG3 | NM_002286.5 | 0.409 | 0.56 | −4.86 | 0.61 | 0.0054 | 0.0009 |
| MRC1 | NM_002438.2 | 0.424 | 0.636 | −6.03 | −0.41 | 0.001 | 0.0096 |
| PDCD1 | NM_005018.1 | 0.314 | 0.442 | −7.49 | −3.44 | 0.006 | 0.0023 |
| PFKFB3 | NM_004566.3 | 0.206 | 0.414 | −2.51 | 0.38 | .0023 | 0.0033 |
| STAT4 | NM_001243835.1 | 0.297 | 0.531 | −5.51 | −1.62 | .0022 | 0.0039 |

### Predictive Genes Using all Patients Data

While the previous analysis was able to identify genes of interest, they were not able to specify if genes were predictive. To determine genes predictive of Vigil® treatment efficacy, Cox proportional hazards model with interaction term was used to analyze data from both Vigil® and placebo patients (n=91). The Cox model included the treatment group, gene and treatment-by gene interaction term.

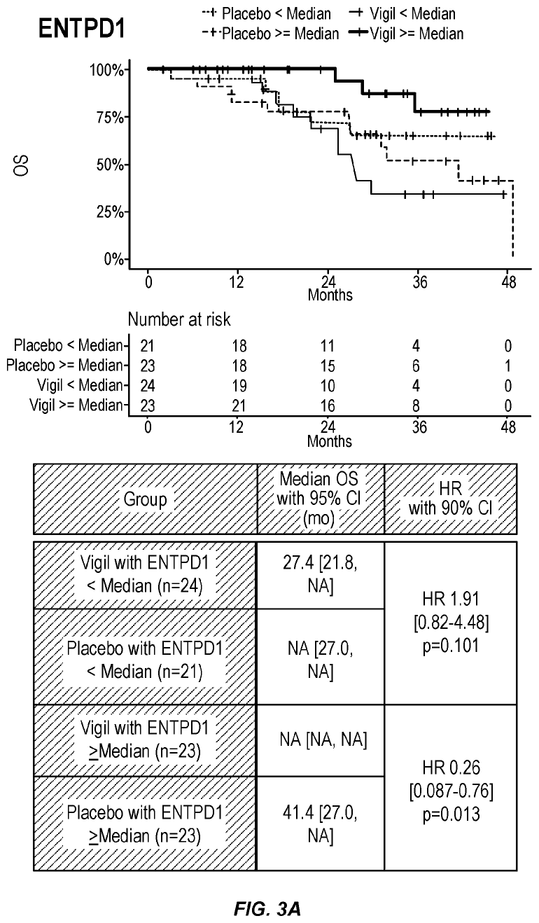

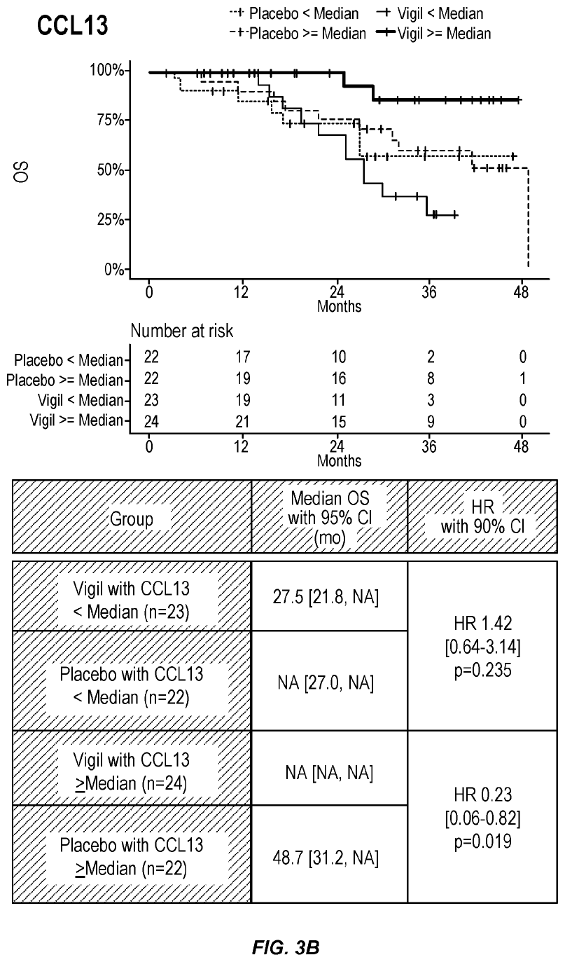

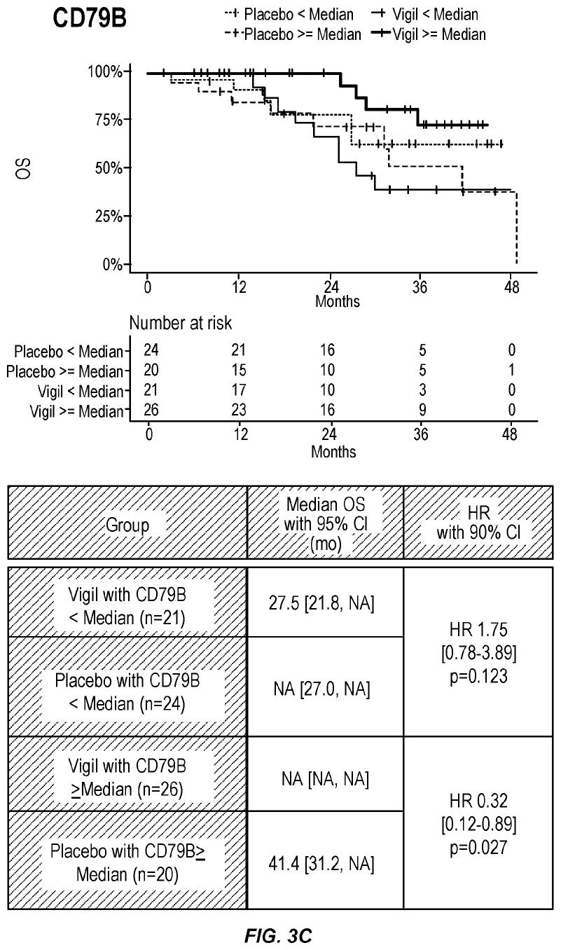

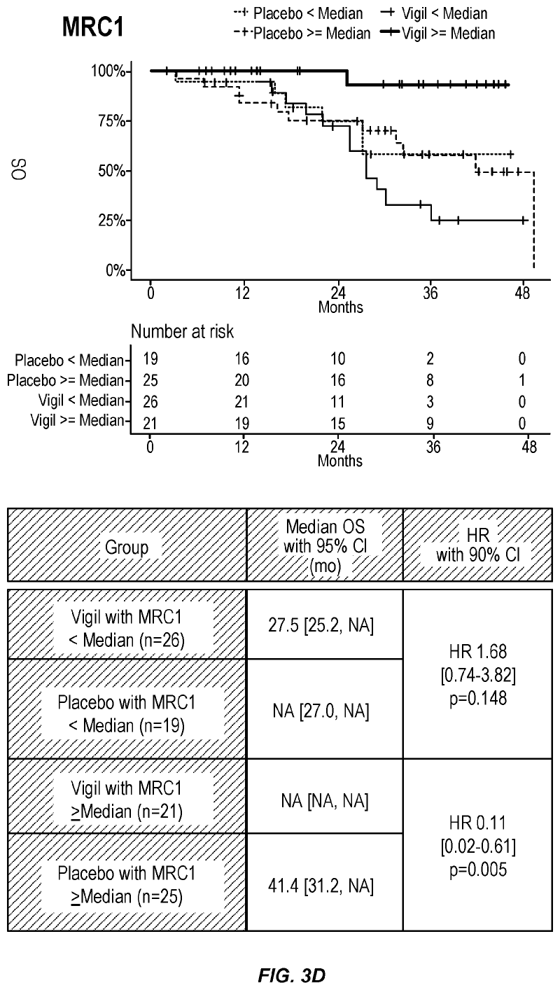

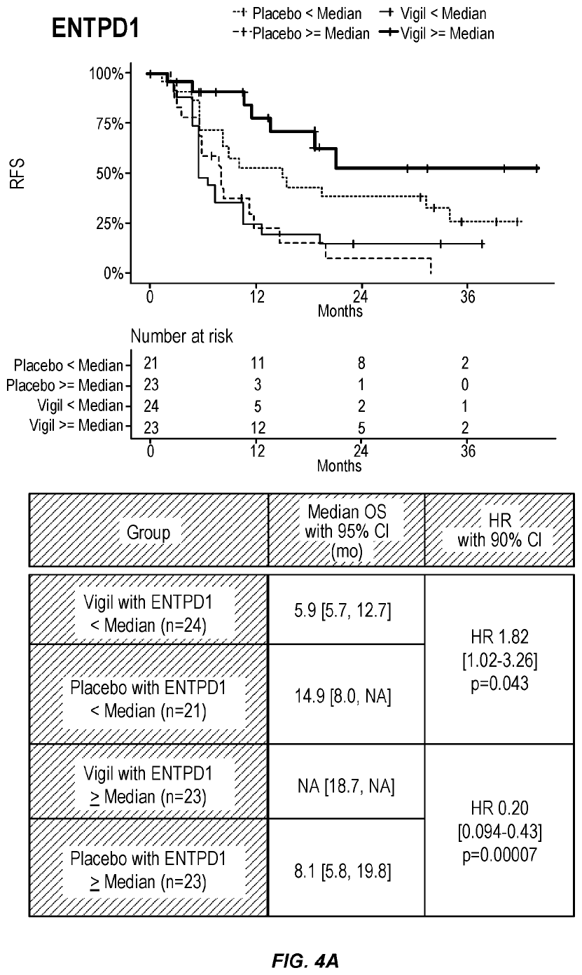

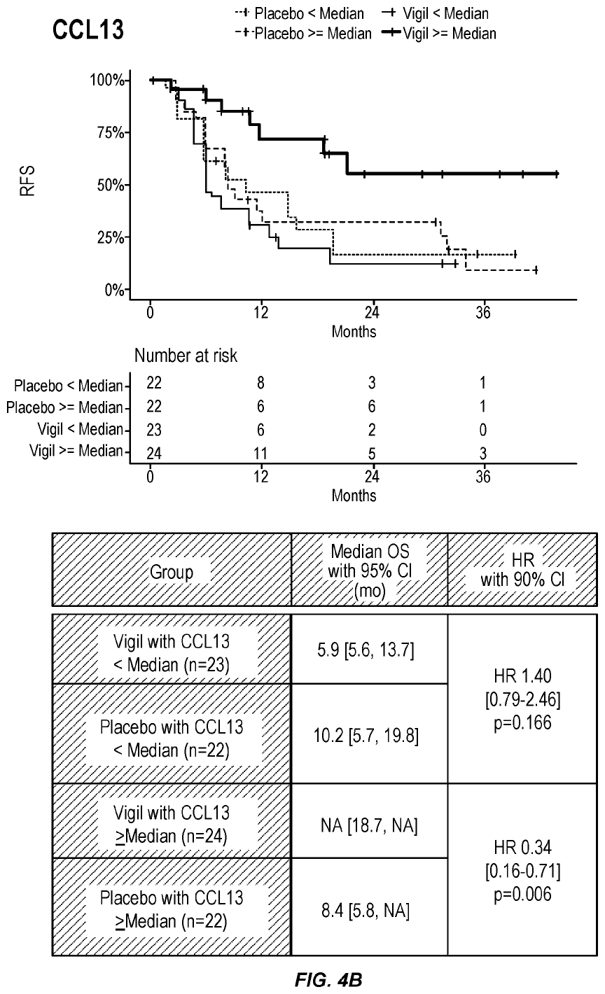

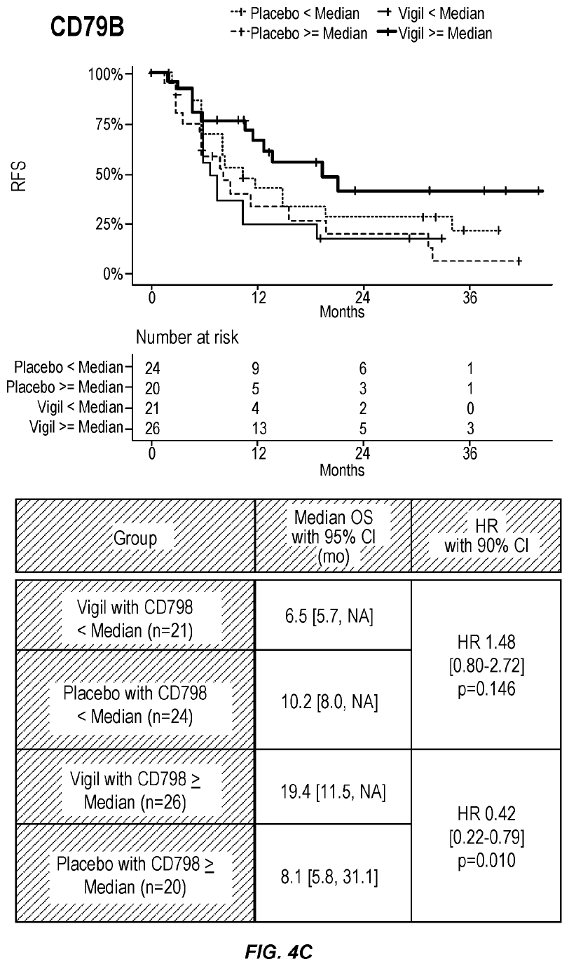

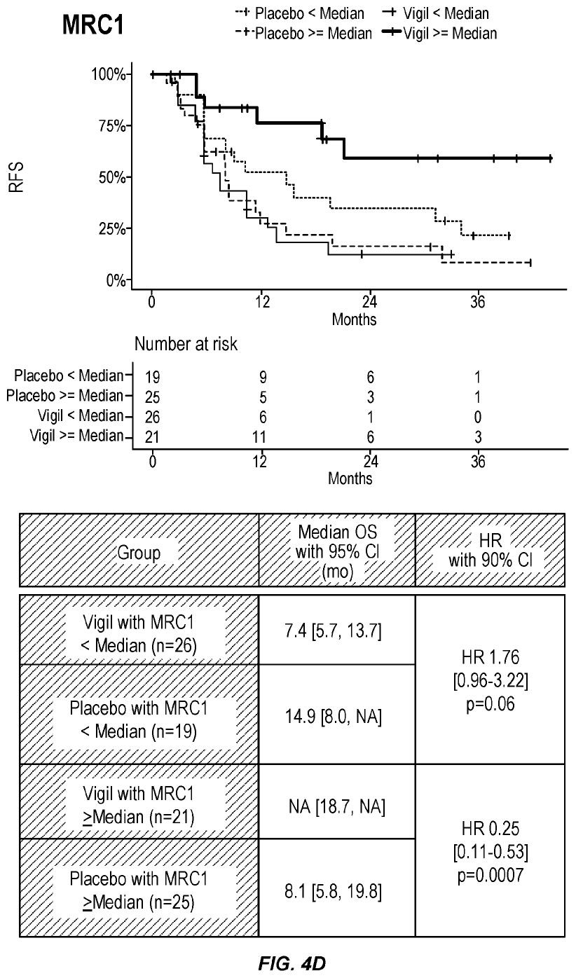

Demographics between Vigil® and placebo were previously shown to not impact clinical benefit results. Four genes were identified as predictive in both Cox models using continuous and binary data for both OS and RFS (CD79B, CCL13, ENTPD1/CD39 and MRC1). Four separate KM curves were generated for each gene in: (1) Vigil® patients with gene expression<median and ≥median; (2) placebo patients with gene expression<median and ≥median; (3) Vigil® patients with gene expression<median and placebo patients<median; and (4) Vigil® patients with gene expression>median and placebo patients>median. KM curves for OS (FIGS. 3A-3D) and RFS (FIGS. 4A-4D) for placebo vs. Vigil® with < or ≥median expression from these 4 genes demonstrate benefit correlation with ≥ median expression. For patients with ≥median ENTPD1/CD39 expression, OS was not achieved compared to placebo OS of 41.4 months (p=0.013) and median RFS was not achieved in Vigil® treated compared to 8.1 months with placebo (p<0.00007). Patients with ≥median expression levels of CCL13, CD79B and MRC1 also demonstrated OS benefit when receiving Vigil® compared to placebo (median not reached vs 48.7 months, p=0.019; not reached vs 41.4 months, p=0.027; not reached vs 41.4, p=0.005 respectively). Similar results demonstrating RFS benefit were observed (not achieved vs 8.4 months p=0.006; 19.4 vs 8.1 months, p=0.010; not achieved vs 8.1 months, p=0.001 respectively). The two-sided p values of the interaction term in the Cox model and one-sided p values of log rank test comparing the OS and RFS KM curves are shown in Table 3 and 4.

TABLE 3
Two-sided p values of the interaction term in the Cox model.

| | Interaction | Interaction | Interaction | Interaction |
|---|---|---|---|---|
| | Term | Term | Term | Term |
| | (continuous)* | (continuous)* | (binary)** | (binary)** |
| | OS | RFS | OS | RFS |
| ENTPD | 0.00751 | 0.00375 | 0.0158 | 0.00014 |
| CCL13 | 0.0190 | 0.00271 | 0.044 | 0.00998 |
| CD79B | 0.00426 | 0.00280 | 0.0303 | 0.0152 |
| MRC1 | 0.01040 | 0.0169 | 0.0173 | 0.000822 |

*Analyzed with genes as raw continuous data.
**Analyzed with genes dichotomized

TABLE 4
One-sided p values of log-rank test comparing two KMs and hazard
ratios and 90% CI from the univariate Cox proportional hazards model
based on four predicted genes from multivariate analysis.

| | Vigil ® ≥ median vs. | | Vigil ® ≥ median vs. | | Vigil ® ≥ median vs. | | Vigil ® ≥ median vs. | |
|---|---|---|---|---|---|---|---|---|
| | Vigil ® < median | | Vigil ® < median | | placebo ≥ median | | placebo ≥ median | |
| | OS | | RFS | | OS | | RFS | |
| | P value | HR | P value | HR | P value | HR | P value | HR |
| ENTPD | 0.002 | 0.177 | 0.0003 | 0.238 | 0.013 | 0.257 | 0.00007 | 0.200 |
| | | [0.059, | | [0.114, | | [0.087, | | [0.094, |
| | | 0.524] | | 0.498] | | 0.761] | | 0.427] |
| CCL13 | 0.0005 | 0.119 | 0.0003 | 0.236 | 0.019 | 0.228 | 0.006 | 0.338 |
| | | [0.034, | | [0.113, | | [0.063, | | [0.161, |
| | | 0.423] | | 0.493] | | 0.824] | | 0.709] |
| CD79B | 0.006 | 0.248 | 0.014 | 0.423 | 0.027 | 0.324 | 0.010 | 0.421 |
| | | [0.092, | | [0.219, | | [0.118, | | [0.224, |
| | | 0.670] | | 0.817] | | 0.892] | | 0.793] |
| MRC1 | 0.0001 | 0.058 | 0.0004 | 0.229 | 0.005 | 0.109 | 0.001 | 0.245 |
| | | [0.010, | | [0.105, | | [0.019, | | [0.112, |
| | | 0.325] | | 0.502] | | 0.613] | | 0.535] |

### Multivariate Analysis Vigil® Patients Only

To further select significant gene associations with OS or RFS in Vigil® treated patients, the my.stepwise.coxph function in R was used as the stepwise variable selection procedure (with iterations between the ‘forward’ and ‘backward’ steps) including the 4 genes showing RFS and OS advantage to Vigil® treatment over placebo. Two common strategies for adding or removing variables in a multiple regression model are backward elimination and forward selection. Backward elimination begins with all genes included in the model and eliminates variables one-by-one until the model cannot be improved per the model fitting criterion. Forward selection starts with no variables included in the model, then adds variables according to importance (e.g. based on p values) until no other significant variables are found. The significance level for variable entry in the model was set at 0.01 and for variable stay was set at 0.01 to account for potential multiplicity in the model selection process. ENTPD1/CD39 was the only gene identified through this stepwise model selection process for both OS and RFS (p value<0.001).

### Subgroup Vigil/Placebo: HRP, ENTPD1/CD39

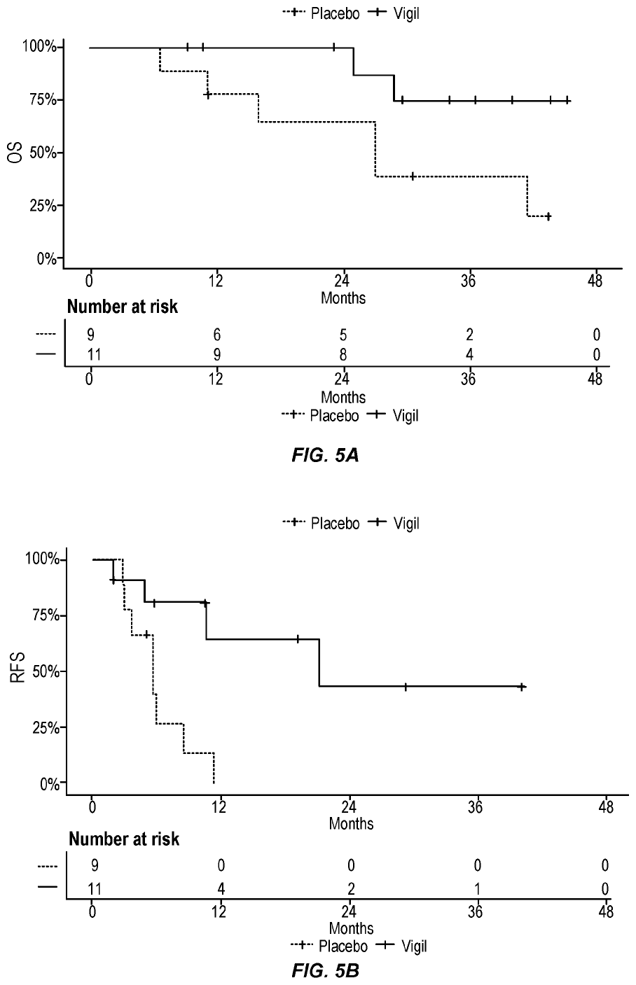

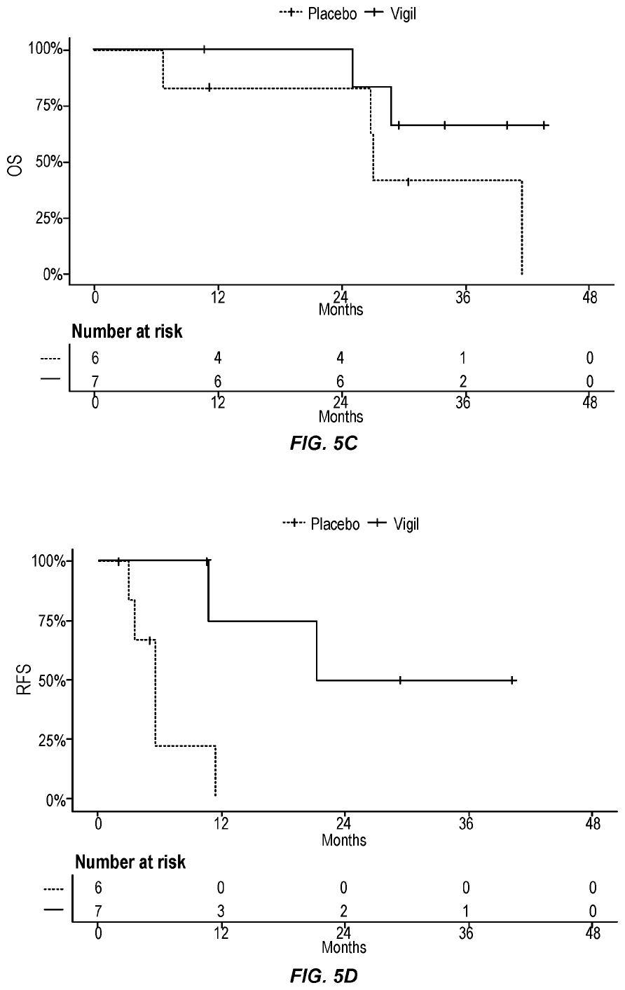

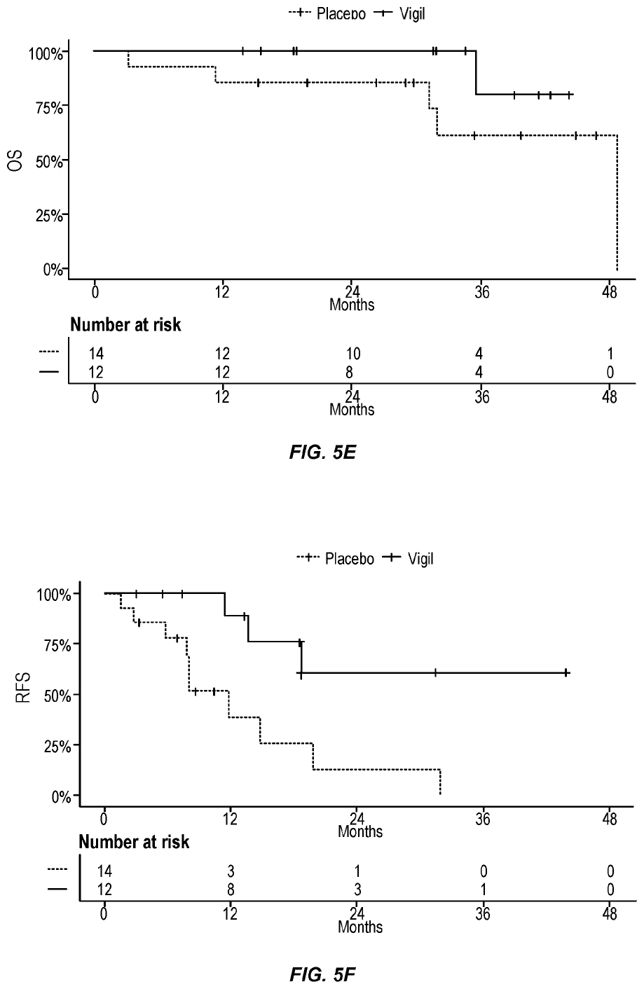

Twenty of the 91 patients (22%) enrolled into the VITAL trial (11 Vigil, 9 placebo) had HRP molecular profile and ENTPD1/CD39 “high” expression. Note HRP status and TP53 mutations have been identified in previous analyses as predictive of Vigil® response. OS advantage was demonstrated (FIG. 5A) in the Vigil® treated HRP/high ENTPD1/CD39 patients relative to placebo (not achieved vs 27 months, HR=0.23, p=0.025). In the same subset, the median RFS for Vigil® was 21.1 months and 5.6 months for placebo (HR=0.18, p=0.004) (FIG. 5B). Despite small sample size, these subgroup results support additional survival benefit in patients whose tumors demonstrate ENTPD1/CD39 high expression in the HRP subgroup. In order to assess the impact of HRP and ENTPD1 on outcomes in patients treated with Vigil, multivariate analyses including HRP status and ENTPD1 as factors were conducted on OS and RFS for all Vigil® patients. For OS, based on the multivariate Cox model for Vigil® patients including both HRP status and ENTPD1 as factors, the p values for HRP status and ENTPD1 status are 0.30 and 0.007 respectively. For RFS, based on the multivariate Cox model for Vigil® patients including both HRP status and ENTPD1 as factors, the p values for HRP status and ENTPD1 status are 0.15 and 0.0005 respectively. This demonstrates that within the Vigil® patients, after adjusting for HRP status, ENTPD1 is still a statistically significant factor. ENTPD1 high Vigil® patients demonstrated improved OS and RFS outcomes compared with ENTPD1 low Vigil® patients.

Subgroup Vigil/Placebo: HRP, p53, ENTPD1/CD39

Evidence of survival advantage was further suggested in patients with tumors demonstrating high ENTPD1/CD39 expression and of HRP/p53mu profile. Despite the small sample size (n=13), a trend toward OS benefit with Vigil® therapy (median not reached vs 27 months, HR=0.34, p=0.099) and robust RFS benefit (21.1 vs 5.6 months, HR=0.09, p=0.004) was suggested (FIGS. 5C and 5D).

### Subgroup Vigil/Placebo: HRD, ENTPD1/CD39

Twenty-six of the 91 patients (29%) had tumors with elevated ENTPD1/CD39 expression that were also HRD (including BRCA1/2-mutation and BRCA1/2-wt/HRD). There appeared to be a trend towards improved OS with Vigil® therapy (median not reached vs 48.7 months, HR=0.24, p=0.08) (FIG. 5E) and RFS difference between Vigil® and placebo was highly significant in this population (median not reached vs 11.8 months, HR=0.21, p=0.005) (FIG. 5F)

### Discussion

Analysis of tumor block material was conducted in order to generate NanoString® PanCancer Immuno-Oncology 360™ molecular profiles involving 750 gene expression signals. Consequently, univariate and multivariate statistical methods as well as stepwise Cox hazards models identified high expression (≥median) of ENTPD1 as a highly significant mRNA signal. Based on data from the VITAL study, ENTPD1 was predictive of OS and RFS benefit with Vigil treatment administered to newly diagnosed Stage IIIb-IV ovarian cancer patients who received Vigil maintenance therapy following debulking surgery and adjuvant chemotherapy compared to placebo. ENTPD1, also known as CD39, functions as a master regulator to maintain the balance between proinflammatory and immunosuppressive regulatory function (10). ENTPD1 protein (CD39) is ubiquitously expressed in the vasculature, B cells, NK cells, dendritic cells, monocytes, macrophages, regulatory T cells and monocyte derived suppressor cells in the TME (30, 31). CD8+ T cells demonstrate T cell exhaustion signatures with malignant upregulation of CD39 in the tumor microenvironment (32-34). Moreover, T regulatory cell upregulation of CD39 within the tumor microenvironment generates immunosuppressive activity thereby facilitating malignant growth and survival (35, 36). Blockage of CD39 in murine cancer models induces anticancer activity and CD39 deficient mice demonstrated a reduction in tumor growth (37-40). In essence, ENTPD1 mRNA expression and upregulation of CD39 protein is associated with tumor growth advantage. However, in our assessment, patients with baseline elevated expression of CD39 demonstrated optimal response following treatment with Vigil compared to low expression and compared to placebo. Particular survival advantage with CD39 expression and Vigil treatment was not only separately demonstrated in the Vigil cohort but also against placebo patients expressing ENTPD1. The primary VITAL study results suggest that Vigil induction of GMCSF, knock down of TGFβ1 and TGFβ2 and induced CD8+ T cell activity targeted to personal cancer neoantigens provide anticancer activity that beneficially impacts OS and RFS in newly diagnosed Stage III/IV ovarian cancer patients receiving Vigil as maintenance therapy. This activity appears to be correlated to high ENTPD1 expression. Interestingly, in a murine model, high levels of TGFβ were associated with immunosuppressive CD39+ myeloid derived suppressor cells (MDSC) (41). Additionally, Li et al (42) demonstrated that MDSCs from patients with NSCLC upregulate CD39 via stimulation with TGFβ, thereby inhibiting T and NK cell activity. One can hypothesize that downregulation of TGFβ expression by Vigil can reduce levels of CD39+ immunosuppressive cells and that this effect might be most impactful in patients with high levels of CD39/ENTPD1 expression at baseline. Interestingly placebo treated patients from the VITAL study with high ENTPD1/CD39 expression tended to show poorer survival compared to patients with lower expression, reflecting the immunosuppressive role of CD39 in these patients, which may be offset with Vigil therapy.

Previously, we demonstrated RFS and OS benefit in the VITAL trial in patients with HRP molecular profile (8, 9). We also suggested DNA mutation (p53mu) may further improve definition of Vigil responsive patients (29). Results of mRNA expression via NanoString® signature also strongly support enhanced benefit in OS and RFS in VITAL trial patients with high ENTPD1 expression receiving Vigil compared to placebo. These results support the need for further verification of ENTPD1 as a biomarker of sensitivity to Vigil treatment in ovarian cancer and possibly other solid tumors with high ENTPD1 expression. Phase III clinical trial assessment of Vigil in combination with bevacizumab against bevacizumab single agent in the HRP population is planned to initiate in 2022. ENTPD1 expression via NanoString® assay will be further assessed in these patients as well.

Results also support therapeutic assessment of combination CD39 inhibition and Vigil as a reasonable direction for clinical testing. CD39 monoclonal antibodies have demonstrated significant anticancer activity in murine models as single agent and in combination with checkpoint inhibitors and autologous EBV-specific human T cells (43). Currently, there are four CD39 targeting agents in early Phase I clinical trials under evaluation. TTX-030 (Tizona, South San Francisco, CA), a human monoclonal antibody against CD39, increased CD4+ and CD8+ T cells in vitro and decreased tumor growth syngeneic in murine models (44). TTX-030 is currently in evaluation in a Phase I study (NCT04306900) in combination with standard of care chemotherapy and pembrolizumab. Another monoclonal CD39 antibody, IPH5201 (Innate Pharma, Marseille, France) showed evidence of dendritic cell, macrophage and effector T cell activation in preclinical models (45). A Phase I study (NCT04261075) is currently investigating IPH5201 as monotherapy or in combination with durvalumab with and without the CD73 inhibitor oleclumab in advanced solid tumors. Surface Oncology (Cambridge, Massachusetts) is also investigating a CD39 monoclonal antibody, SRF617, which is currently in Phase I study (NCT04336098) in advanced solid tumors as monotherapy or in combination with gemcitabine, paclitaxel and pembrolizumab. SRF617 in combination with immunotherapy demonstrated improved survival in murine models (46). Finally, ES002023 (Elpiscience Biopharma, Pudong, China) is also entering a Phase I (NCT05075564) trial in advanced solid tumors (Part 1) and pancreatic ductal adenocarcinoma, NSCLC and colorectal cancer (Part 2) as monotherapy. Vigil combination with CD39 inhibition may also provide a fruitful direction for further combination therapy.

It is also possible that in a larger patient population receiving Vigil, evidence of other immune modulatory pathways identified by NanoString® analysis (i.e., CXCL13 (47, 48), CD79B (49), MRC1 (50)) will demonstrate statistical correlation with OS and RFS in certain patient subpopulations. Such results may further direct research towards combination therapeutic approaches with Vigil which may optimize Vigil combination therapeutic management to various molecular biomarkers expression patterns that are relevant to specific combinations of signal sensitivity. In addition, the my.stepwise.coxph function in R is particularly useful to refine correlations when a large number of genes have been identified. Further testing of Vigil in combination with other relevant immune modulators given Vigil's differentiated mechanism of action and safety profile are underway and may be further refined on the basis of future bioinformatics work of this nature.

Molecular biomarker assessment to define optimally responsive patients to Vigil therapy will involve more comprehensive analysis of p53mu, BRCA1/2-wt, ENTPD1 and HRP molecular profile as part of further clinical investigation of Vigil. Evidence provided here suggests these signals can act independently in defining sensitive subpopulations but also appear to support evidence of synergistic activity when biomarkers are assessed in combination. These multivariate analyses will continue to help identify optimal subpopulations with potential to benefit via treatment with Vigil. Moreover, results combining DNA and mRNA expression pathways may optimize direction of Vigil combination studies. Results also justify trial consideration of Vigil in other solid tumor patients with HRP profile, p53mu and those with ENTPD1 high expression by NanoString® PanCancer Immuno-Oncology 360™ CodeSet analysis.

In conclusion, this analysis identified gene signatures associated with Vigil response when used as frontline maintenance therapy in newly diagnosed ovarian cancer patients. Interestingly, ENTPD1/CD39, a gene associated with an immunosuppressive tumor microenvironment, was most highly predictive of Vigil responsiveness. Previous work has indicated TGFβ may upregulate CD39 in immunosuppressive myeloid cells, such that Vigil's effect in downregulating TGFβ may counter this effect and account for its activity in patients with high expression of CD39. Combining previously identified biomarkers of Vigil response, such as HRP and p53 mutational status, with ENTPD1 expression may allow for refined identification of Vigil responsive populations. These results will be confirmed and built upon in subsequent clinical trials assessing Vigil as a treatment for solid tumors.

### REFERENCES

1 Ledermann, J. A. et al. LBA 25-Scientific Plenary: Avelumab in combination with and/or following chemotherapy vs chemotherapy alone in patients with previously untreated epithelial ovarian cancer: Results from the phase 3 javelin ovarian 100 trial. Gynecologic Oncology 159, 13-14 (2020).
2 Moore, K. N. et al. Atezolizumab, Bevacizumab, and Chemotherapy for Newly Diagnosed Stage III or IV Ovarian Cancer: Placebo-Controlled Randomized Phase III Trial (IMagyn050/GOG 3015/ENGOT-OV39). J Clin Oncol 39, 1842-1855, doi: 10.1200/JCO.21.00306 (2021).
3 Matulonis, U. A. et al. Antitumor activity and safety of pembrolizumab in patients with advanced recurrent ovarian cancer: results from the phase II KEYNOTE-100 study. Ann Oncol 30, 1080-1087, doi: 10.1093/annonc/mdz135 (2019).
4 Sabbatini, P. et al. Abagovomab as maintenance therapy in patients with epithelial ovarian cancer: a phase III trial of the AGO OVAR, COGI, GINECO, and GEICO—the MIMOSA study. J Clin Oncol 31, 1554-1561, doi: 10.1200/JCO.2012.46.4057 (2013).
Vergote, I. et al. A Randomized, Double-Blind, Placebo-Controlled, Phase III Study to Assess Efficacy and Safety of Weekly Farletuzumab in Combination With Carboplatin and Taxane in Patients With Ovarian Cancer in First Platinum-Sensitive Relapse. J Clin Oncol 34, 2271-2278, doi: 10.1200/JCO.2015.63.2596 (2016).
6 Monk, B. J. et al. A phase 2, randomized, double-blind, placebo-controlled study of chemo-immunotherapy combination using motolimod with pegylated liposomal doxorubicin in recurrent or persistent ovarian cancer: a Gynecologic Oncology Group partners study. Ann Oncol 28, 996-1004, doi: 10.1093/annonc/mdx049 (2017).
7 Takenaka, M. C., Robson, S. & Quintana, F. J. Regulation of the T Cell Response by CD39. Trends Immunol 37, 427-439, doi: 10.1016/j.it.2016.04.009 (2016).
8 Senzer, N. et al. Long Term Follow Up: Phase I Trial of “bi-shRNA furin/GMCSF DNA/Autologous Tumor Cell” Immunotherapy (FANG™) in Advanced Cancer. Journal of Vaccines & Vaccination 4, 209 (2013).
9 Johnson, D. B. et al. Targeted Next Generation Sequencing Identifies Markers of Response to PD-1 Blockade. Cancer Immunol Res 4, 959-967, doi: 10.1158/2326-6066.CIR-16-0143 (2016).
10 Maples, P. et al. FANG Vaccine: Autologous Tumor Cell Vaccine Genetically Modified to Express GM-CSF and Block Production of Furin. BioProcessing Journal 8, 4-14, doi: 10.12665/J84.Maples (2010).
11 Senzer, N. et al. Phase I trial of “bi-shRNAi (furin)/GMCSF DNA/autologous tumor cell” vaccine (FANG) in advanced cancer. Mol Ther 20, 679-686, doi: 10.1038/mt.2011.269 (2012).
12 Rocconi, R. P. et al. Gemogenovatucel-T (Vigil) immunotherapy as maintenance in frontline stage III/IV ovarian cancer (VITAL): a randomised, double-blind, placebo-controlled, phase 2b trial. Lancet Oncol 21, 1661-1672, doi: 10.1016/S1470-2045 (20) 30533-7 (2020).
13 Rocconi, R. P. et al. Gemogenovatucel-T (Vigil) immunotherapy demonstrates clinical benefit in homologous recombination proficient (HRP) ovarian cancer. Gynecol Oncol 161, 676-680, doi: 10.1016/j.ygyno.2021.03.009 (2021).
14 Walter, A. et al. Gemogenovatucel-T (Vigil) maintenance immunotherapy: 3-year survival benefit in homologous recombination proficient (HRP) ovarian cancer. Gynecologic Oncology 163, 459-464, doi: 10.1016/j.ygyno.2021.10.004 (2021).
15 Oh, J. et al. Long-term follow-up of Phase 2A trial results involving advanced ovarian cancer patients treated with Vigil® in frontline maintenance. Gynecol Oncol Rep 34, 100648, doi: 10.1016/j.gore.2020.100648 (2020).
16 Morand, S., Devanaboyina, M., Staats, H., Stanbery, L. & Nemunaitis, J. Ovarian Cancer Immunotherapy and Personalized Medicine. Int J Mol Sci 22, doi: 10.3390/ijms22126532 (2021).
17 Keenan, T. E., Burke, K. P. & Van Allen, E. M. Genomic correlates of response to immune checkpoint blockade. Nat Med 25, 389-402, doi: 10.1038/s41591-019-0382-x (2019).
18 Choucair, K. et al. TMB: a promising immune-response biomarker, and potential spearhead in advancing targeted therapy trials. Cancer Gene Ther 27, 841-853, doi: 10.1038/s41417-020-0174-y (2020).
19 Kandalaft, L. E., Powell, D. J., Jr., Singh, N. & Coukos, G. Immunotherapy for ovarian cancer: what's next? J Clin Oncol 29, 925-933, doi: 10.1200/JCO.2009.27.2369 (2011).
20 Rocconi, R. P. et al. Long-Term Follow-Up of Gemogenovatucel-T (Vigil) Survival and Molecular Signals of Immune Response in Recurrent Ovarian Cancer. Vaccines (Basel) 9, 894, doi: 10.3390/vaccines9080894 (2021).
21 Sliheet, E. et al. Network based analysis identifies TP53m-BRCA1/2wt-homologous recombination proficient (HRP) population with enhanced susceptibility to Vigil immunotherapy. Cancer Gene Therapy, doi: 10.1038/s41417-021-00400-x (2021).
22 Company, I. H. S. C. (2017).
23 Hausler, S. F. et al. Anti-CD39 and anti-CD73 antibodies A1 and 7G2 improve targeted therapy in ovarian cancer by blocking adenosine-dependent immune evasion. Am J Transl Res 6, 129-139 (2014).
24 Clayton, A., Al-Taei, S., Webber, J., Mason, M. D. & Tabi, Z. Cancer exosomes express CD39 and CD73, which suppress T cells through adenosine production. J Immunol 187, 676-683, doi: 10.4049/jimmunol. 1003884 (2011).
25 Koziak, K., Sevigny, J., Robson, S. C., Siegel, J. B. & Kaczmarek, E. Analysis of CD39/ATP diphosphohydrolase (ATPDase) expression in endothelial cells, platelets and leukocytes. Thromb Haemost 82, 1538-1544 (1999).
26 Antonioli, L., Pacher, P., Vizi, E. S. & Haskó, G. CD39 and CD73 in immunity and inflammation. Trends Mol Med 19, 355-367, doi: 10.1016/j.molmed.2013.03.005 (2013).
27 Thelen, M., Lechner, A., Wennhold, K., von Bergwelt-Baildon, M. & Schlosser, H. A. CD39 Expression Defines Cell Exhaustion in Tumor-Infiltrating CD8 (+) T Cells-Letter. Cancer Res 78, 5173-5174, doi: 10.1158/0008-5472.CAN-18-0873 (2018).
28 Canale, F. P. et al. CD39 expression defines cell exhaustion in tumor-infiltrating CD8+ T cells. Cancer research 78, 115-128 (2018).
29 Simoni, Y. et al. Bystander CD8 (+) T cells are abundant and phenotypically distinct in human tumour infiltrates. Nature 557, 575-579, doi: 10.1038/s41586-018-0130-2 (2018).
30 Borsellino, G. et al. Expression of ectonucleotidase CD39 by Foxp3+ Treg cells: hydrolysis of extracellular ATP and immune suppression. Blood 110, 1225-1232, doi: 10.1182/blood-2006-12-064527 (2007).
31 Gu, J. et al. Human CD39 hi regulatory T cells present stronger stability and function under inflammatory conditions. Cellular & molecular immunology 14, 521-528 (2017).
32 Sun, X. et al. CD39/ENTPD1 expression by CD4+Foxp3+ regulatory T cells promotes hepatic metastatic tumor growth in mice. Gastroenterology 139, 1030-1040, doi: 10.1053/j.gastro.2010.05.007 (2010).
33 Feng, L. et al. Vascular CD39/ENTPD1 directly promotes tumor cell growth by scavenging extracellular adenosine triphosphate. Neoplasia 13, 206-216, doi: 10.1593/neo. 101332 (2011).
34 Sun, X. et al. Disordered purinergic signaling and abnormal cellular metabolism are associated with development of liver cancer in Cd39/ENTPD1 null mice. Hepatology 57, 205-216, doi: 10.1002/hep.25989 (2013).
35 Zhang, H. et al. The role of NK cells and CD39 in the immunological control of tumor metastases. Oncoimmunology 8, e1593809, doi: 10.1080/2162402X.2019.1593809 (2019).
36 Ryzhov, S. V. et al. Role of TGF-beta signaling in generation of CD39+CD73+ myeloid cells in tumors. J Immunol 193, 3155-3164, doi: 10.4049/jimmunol. 1400578 (2014).
37 Herron, J. et al. Vigil: Personalized Immunotherapy Generating Systemic Cytotoxic T cell Response. Cancer Sci. Res 1, 210-221 (2020).
38 Li, X. Y. et al. Targeting CD39 in Cancer Reveals an Extracellular ATP- and Inflammasome-Driven Tumor Immunity. Cancer Discov 9, 1754-1773, doi: 10.1158/2159-8290.CD-19-0541 (2019).
39 Lerner, A. G. et al. Abstract 5012: Targeting CD39 with a first-in-class inhibitory antibody prevents ATP processing and increases T-cell activation. Cancer Research 79, 5012, doi: 10.1158/1538-7445.Am2019-5012 (2019).
40 Perrot, I. et al. Blocking Antibodies Targeting the CD39/CD73 Immunosuppressive Pathway Unleash Immune Responses in Combination Cancer Therapies. Cell Reports 27, 2411-2425.e2419, doi: 10.1016/j.celrep.2019.04.091 (2019).
41 Das, S. G. et al. Abstract 6639: SRF617, a potent enzymatic inhibitor of CD39, demonstrates single-agent activity and cooperates with various cancer therapies in both solid tumor and hematologic malignancies. Cancer Research 80, 6639-6639, doi: 10.1158/1538-7445.Am2020-6639 (2020).
42 Kazanietz, M. G., Durando, M. & Cooke, M. CXCL13 and Its Receptor CXCR5 in Cancer: Inflammation, Immune Response, and Beyond. Front Endocrinol (Lausanne) 10, 471, doi: 10.3389/fendo.2019.00471 (2019).
43 Zhu, D., Ye, W. & Jiang, J. Clinical significance of CXCL13/CXCR5 axis in human cancers. Translational Cancer Research 7, 1737-1742 (2018).
44 Ferreri, A. J. M. Targeted therapies make room, anti-CD79b agents are coming. Lancet Oncol 20, 898-900, doi: 10.1016/S1470-2045 (19) 30182-2 (2019).
45 von Ehr, A. et al. Inhibition of Microglial TGFbeta Signaling Increases Expression of Mrc1. Front Cell Neurosci 14, 66, doi: 10.3389/fncel.2020.00066 (2020).
46 Jaynes, J. M. et al. Mannose receptor (CD206) activation in tumor-associated macrophages enhances adaptive and innate antitumor immune responses. Sci Transl Med 12, doi: 10.1126/scitranslmed.aax6337 (2020).
47 Yang, M. et al. CXCL13 shapes immunoactive tumor microenvironment and enhances the efficacy of PD-1 checkpoint blockade in high-grade serous ovarian cancer. Journal for Immuno Therapy of Cancer 9, e001136, doi: 10.1136/jitc-2020-001136 (2021).
48 Kinker, G. S. et al. B Cell Orchestration of Anti-tumor Immune Responses: A Matter of Cell Localization and Communication. Frontiers in Cell and Developmental Biology 9, doi: 10.3389/fcell.2021.678127 (2021).

All publications, patents and patent applications cited in this specification are herein incorporated by reference as if each individual publication, patent or patent application were specifically and individually indicated to be incorporated by reference. Although the foregoing disclosure has been described in some detail by way of illustration and example for purposes of clarity of understanding, it will be readily apparent to those of ordinary skill in the art that, in light of the teachings of this application, that certain changes and modifications may be made thereto without departing from the spirit or scope of the appended claims.

1. A method for treating an individual having cancer, the method comprising:
profiling an expression level of one or more genes selected from the group consisting of ENTPD1, CCL13, CD79B, and MRC1, in a tumor obtained from said individual;
determining the presence of an elevated expression level of the one or more genes selected from the group consisting of ENTPD1, CCL13, CD79B, and MRC1 in the tumor obtained from said individual; and
administering to the individual a therapy comprising an expression vector having (i) a first insert comprising a nucleic acid sequence encoding a Granulocyte Macrophage Colony Stimulating Factor (GM-CSF) sequence; and (ii) a second insert,
wherein the second insert encodes one or more bifunctional short hairpin RNAs (shRNA) capable of hybridizing to one of more regions of a mRNA transcript encoding furin, wherein at least one of the regions is selected from base sequences 300-318, 731-740, 1967-1991, 2425-2444, 2827-2851 or 2834-2852 of SEQ ID NO: 1, thereby inhibiting furin expression via RNA interference,
wherein each bifunctional short hairpin RNA comprises a first stem-loop structure that comprises an siRNA component and a second stem-loop structure that comprises a miRNA component and wherein the shRNA incorporates siRNA (cleavage dependent) and miRNA (cleavage-independent) motifs,
wherein the therapy improves the treatment response in the individual with the elevated expression level compared to a patient with a tumor having a low expression level of the gene.

2. The method of claim 1, wherein the second insert comprises two stem-loop structures each with a miR-30a loop, and the first stem-loop structure has complete complementary guiding strand and passenger strand, while the second stem-loop structure has three basepair (bp) mismatches at positions 9 to 11 of the passenger strand.

3. The method of claim 1, wherein the second insert comprises a sequence having at least 90% identity to the sequence of SEQ ID NO:2.

4. (canceled)

5. The method of claim 1, wherein the gene is ENTPD1.

6-8. (canceled)

9. The method of claim 1, wherein the cancer is selected from the group consisting of ovarian cancer, breast cancer, melanoma, and lung cancer.

10. (canceled)

11. The method of claim 1, wherein the elevated expression level of the one or more genes means greater than or equal to the median of the expression level of the gene in individuals having the cancer.

12. The method of claim 1, wherein the low expression level of the gene means less than the median of the expression level of the gene in individuals having the cancer.

13. The method of claim 1, wherein the method further comprises determining the genotypes of at least two genes selected from the group consisting of BRCA1, BRCA2, and TP53.

14. (canceled)

15. A method for predicting responsiveness of an individual having or suspected of having cancer to a therapy comprising an expression vector having (i) a first insert comprising a nucleic acid sequence encoding a Granulocyte Macrophage Colony Stimulating Factor (GM-CSF) sequence; and (ii) a second insert, the method comprising:
profiling the expression level of one or more genes selected from the group consisting of ENTPD1, CCL13, CD79B, and MRC1, in a tumor obtained from said individual;
determining the presence of an elevated expression level of the one or more genes selected from the group consisting of ENTPD1, CCL13, CD79B, and MRC1; and
identifying the individual with the elevated expression level of the one or more genes as predicted to have an increased responsiveness to the therapy, compared to a patient with a tumor having a low expression of the gene,
wherein the second insert encodes one or more bifunctional short hairpin RNAs (shRNA) capable of hybridizing to one of more regions of a mRNA transcript encoding furin, wherein at least one of the regions is selected from base sequences 300-318, 731-740, 1967-1991, 2425-2444, 2827-2851 or 2834-2852 of SEQ ID NO:1, thereby inhibiting furin expression via RNA interference, and
wherein each bifunctional short hairpin RNA comprises a first stem-loop structure that comprises an siRNA component and a second stem-loop structure that comprises a miRNA component and wherein the shRNA incorporates siRNA (cleavage dependent) and miRNA (cleavage-independent) motifs.

16. The method of claim 15, wherein the second insert comprises two stem-loop structures each with a miR-30a loop, and the first stem-loop structure has complete complementary guiding strand and passenger strand, while the second stem-loop structure has three basepair (bp) mismatches at positions 9 to 11 of the passenger strand.

17. The method of claim 15, wherein the second insert comprises a sequence having at least 90% identity to the sequence of SEQ ID NO:2.

18. (canceled)

19. The method of claim 15, wherein the gene is ENTPD1.

20-24. (canceled)

25. The method of claim 15, wherein the elevated expression level of the one or more genes means greater than or equal to the median of the expression level of the gene in individuals having the cancer; and/or
wherein the low expression level of the gene means less than the median of the expression level of the gene in individuals having the cancer.

26. (canceled)

27. The method of claim 15, wherein the method further comprises determining the genotypes of at least two genes selected from the group consisting of BRCA1, BRCA2, and TP53.

28. (canceled)

29. A method for selecting an individual having cancer to be subjected to a therapy comprising an expression vector having (i) a first insert comprising a nucleic acid sequence encoding a Granulocyte Macrophage Colony Stimulating Factor (GM-CSF) sequence; and (ii) a second insert, the method comprising:
profiling the expression level of one or more genes selected from the group consisting of ENTPD1, CCL13, CD79B, and MRC1, in a tumor obtained from said individual;
determining the presence of an elevated expression level of the one or more genes selected from the group consisting of ENTPD1, CCL13, CD79B, and MRC1; and
selecting the individual who is determined to have the elevated expression level of the one or more genes compared to a patient with a tumor having a low expression of the gene as an individual to be subjected to the therapy,
wherein the second insert encodes one or more bifunctional short hairpin RNAs (shRNA) capable of hybridizing to one of more regions of a mRNA transcript encoding furin, wherein at least one of the regions is selected from base sequences 300-318, 731-740, 1967-1991, 2425-2444, 2827-2851 or 2834-2852 of SEQ ID NO: 1, thereby inhibiting furin expression via RNA interference, and
wherein each bifunctional short hairpin RNA comprises a first stem-loop structure that comprises an siRNA component and a second stem-loop structure that comprises a miRNA component and wherein the shRNA incorporates siRNA (cleavage dependent) and miRNA (cleavage-independent) motifs.

30. The method of claim 29, wherein the second insert comprises two stem-loop structures each with a miR-30a loop, and the first stem-loop structure has complete complementary guiding strand and passenger strand, while the second stem-loop structure has three basepair (bp) mismatches at positions 9 to 11 of the passenger strand.

31. The method of claim 29, wherein the second insert comprises a sequence having at least 90% identity to the sequence of SEQ ID NO:2.

32-38. (canceled)

39. The method of claim 29, wherein the elevated gene expression levels of the one or more genes means greater than or equal to the median of the expression level of the gene in individuals having the cancer; and/or
wherein the low expression level of the gene means less than the median of the expression level of the gene in individuals having the cancer.

40. (canceled)

41. (canceled)

42. The method of claim 1, wherein the method comprises determining the status of homologous recombination deficiency (HRD) in the individual.

43. (canceled)

44. A method for identifying genes that are predictive of responsiveness of an individual having or suspected of having cancer to a therapy, the method comprising:
(a) applying a univariate Cox model to Z-scores of a plurality of genes as a continuous variable for both overall survival (OS) and relapse free survival (RFS) in a cohort of patients treated with the therapy to generate a p-value and a corresponding 95% confidence interval (CI) for each of the plurality of genes;
(b) determining whether each gene identified in step (a) is predictive of a treatment advantage of the therapy by analyzing data of both the therapy cohort and a cohort of patients treated with a placebo by using a Cox proportional hazards model to determine if the interaction term between the gene and the treatment cohort was significant to yield predictive genes; and
(c) applying a further univariant Cox model to the identified predictive genes in step (b) in the treated cohort to further refine identification of relevant genes.

45-52. (canceled)
